(19) United States
(12) Patent Application Publication
(10) Pub. No.: US 2013/0274297 A1
(43) Pub. Date: Oct. 17, 2013

(54) PHARMACEUTICAL COMPOSITES OF POORLY WATER SOLUBLE DRUGS AND POLYMERS

(71) Applicants: Paolo Gatti, Sesto San Giovanni (IT); Italo Colombo, Treviglio (IT); Dario Gervasoni, Carugate (IT); Vincenza Pironti, Cavenago Di Brianza (IT); Lia Alborghetti, Scanzorosciate (IT); Flavio Fabiani, Ronco Briantino (IT); Luigi Boltri, Agrate Brianza (IT)

(72) Inventors: Paolo Gatti, Sesto San Giovanni (IT); Italo Colombo, Treviglio (IT); Dario Gervasoni, Carugate (IT); Vincenza Pironti, Cavenago Di Brianza (IT); Lia Alborghetti, Scanzorosciate (IT); Flavio Fabiani, Ronco Briantino (IT); Luigi Boltri, Agrate Brianza (IT)

(73) Assignee: APTALIS PHARMA LIMITED, Bray, Co. Wicklow (IE)

(21) Appl. No.: 13/995,396

(22) PCT Filed: Dec. 22, 2011
(86) PCT No.: PCT/EP2011/073782

§ 371 Date: Jun. 18, 2013

(30) Foreign Application Priority Data
Dec. 22, 2010 (IE) 2010/0799

(51) Int. Cl. A61K 31/4422; A61K 31/18; A61K 31/216

(52) U.S. Cl. CPC A61K 31/4422; A61K 31/216; A61K 31/18
USPC 514/356; 514/543; 514/605

(57) ABSTRACT

The invention provides a composite of drug, polymeric carrier and at least one not cross linked polymer useful to improve the solubility of poorly water soluble drugs. The present invention comprises manufacturing process of this composite material. The manufacturing process is carried out by the solvent induced activation process, wherein the not cross-linked polymer is loaded into the composite from organic solution, possibly together with the drug. Pharmaceutical compositions comprising said composite in combination with pharmaceutical acceptable excipients are also described herein.

### BACKGROUND OF THE INVENTION

The preferred route of drug administration is oral; however, in order for a drug to be effective and to provide the desired clinical response once administered by this route, it must be able to dissolve and to be absorbed in the gastro-intestinal tract. Therefore, drugs with low water solubility are usually also poorly bioavailable upon oral administration, that means they reach the blood stream in very limited amount. For this reason, oral delivery of poorly soluble drugs has become, in the last years, one of the most challenging problems for advanced pharmaceutical research. In fact, it has been calculated that approximately 40% of the existing drugs and more than 50% of all New Chemical Entities are insoluble or poorly soluble in water and may have inherent absorption problems.

A Biopharmaceutics Classification System (BCS) has been proposed by Amidon et al. and accepted by the FDA guidelines for classifying drugs based on recognizing that drug dissolution and gastrointestinal permeability are fundamental parameters governing rate and extent of drug absorption (FIG. 1). According to the BCS, a Class II compound is defined as having low solubility and high permeability where solubility or dissolution rate is limiting in general or on regional basis throughout the GI tract the drug absorption.

Many technological approaches have been developed to address the specific challenges of Class II drugs by reducing the interaction energy barrier for the dissolution. These approaches include micronisation, inclusion of surfactants, formulation of emulsions or microemulsions, use of complexing agents (i.e. cyclodextrins) or creation of high-energy states.

The technology, commercially known as Biorise Technology, is a platform for bioavailability enhancement of poorly soluble drugs. By this technology solubility and dissolution rate are improved by breaking down the drug crystal lattice to get thermodynamically activated forms, amorphous and/or nanocrystalline, stabilized in a biologically inert carrier. This causes a strong reduction of the interaction energy barrier necessary to reach the dissolution of the drug. In fact, the amorphous phase can be considered as a “solid solution” of single drug molecules in the carrier, readily solvated by the water molecules and diffused into the solvent (dissolution). Nanocrystalline drug forms are small in size and are dispersed into the pore network of the carrier. This particular thermodynamic state of nanocrystals results in a strong improvement of the drug dissolution properties.

The change of thermodynamic state of the drug (also called activation) in Biorise technology is accomplished by two different approaches: HEMA (High Energy Mechanochemical Activation) and SIA (Solvent Induced Activation). These two techniques allow drug dispersion inside a proper carrier (eg, polymers, cyclodextrins) using, respectively, mechanical and chemical energy.

The HEMA process is a physical reaction (in absence of solvents) carried out in a high energy mechano-chemical reactor (mill) and involving repeated microfusion, fracturing and comminution of the powder particles. For this reason, the process is called High Energy Mechano-chemical Activation (HEMA). Mechano-chemical activation allows the production of macroscopically homogeneous material starting from powder mixtures. Mechano-chemical activation is capable of forming stable and metastable phases, including oversaturated solid solutions, nanocrystalline (nanometer dimensions), quasi-crystalline states and amorphous phases.

The Solvent Induced Activation (SIA) process, whereby the drug is dissolved in an appropriate solvent (process solvent), loaded onto a cross-linked polymer carrier by swelling and, following removal of the process solvent, produces a dried material containing drug(s) in activated form(s) (amorphous and/or nanocrystalline).

The loading of drugs into cross-linked polymers is a way to molecularly disperse drug particles throughout the macromolecular network of the polymer, leading to an improved solubility pattern.

The stability of compounds prepared with bioavailability enhancement technologies is a prevalent concern. With the Biorise technology, activated drugs loaded onto the carriers have a high physical stability (maintenance of the thermodynamically activated states). A strong interaction between drug and carrier is given by the entrapment of the molecular or nanocrystalline drug dispersion in the polymeric network, which results in a stabilization of the physical states.

An important peculiar aspect of the Biorise technology is that the chemical nature of the drug and the carrier is not affected by the activation process. This means that if drug and carrier are approved for human use, the same will be true for the Biorise prepared system that can be viewed as composite material representing a New Physical Entity instead of a New Chemical Entity.

Known composites consist of a drug and a carrier (two components), they are named binary composite. Biorise binary composites are widely disclosed in previous Biorise patents (EP364944, EP446753). The level of activation of the drug in binary composites depends on interactions between drug and carrier and usually higher activation level is obtained reducing the composite drug load. Maximum level of activation is represented by transition of all the drug into the composite to amorphous form; fully nanocrystalline drug is a lower level of activation compared to fully amorphous.

Even if the drug in the binary composites is in activated form, frequently the activation level is herein not maximized. Moreover, diluted composition (low dosage strength) have to be used with binary composite to maintain a reasonable activation of the drug, but diluted drug loads could be sometimes not sufficient for the production of oral solid dosage forms with therapeutically effective strength (100-200 mg or more). Finding the way to maximize the activation level (i.e. 100% amorphous drug) and to increase the drug load while maintaining high activation are important improvements.

### SUMMARY OF THE INVENTION

To achieve these and other objects, and to meet these and other needs, and in view of its purposes, the present invention relates to a pharmaceutical composite useful to improve the solubility of poorly water soluble drugs through the formation of highly activated solid form of the active ingredient (i.e. amorphous, nano-crystalline etc.). In particular, the invention relates to a ternary composite comprising at least one poorly soluble drug, at least one polymeric carrier and at least one not chemically cross-linked polymer, which is both soluble in water and organic solvent.

The present invention comprises also pharmaceutical composition comprising the composite and pharmaceutically acceptable excipients.

Moreover, the invention provides a process for manufacturing the composite. The process is based on the SIA technology, wherein the not chemically cross-linked polymer, which is both soluble in water and in organic solvent, is loaded into the polymeric carrier from organic solution.

The composite, being formed of three types of components (drug, polymeric carrier, not chemically cross-linked polymer, which is both soluble in water and organic solvent) are named ternary composites to distinguish from those obtained with the known Biorise technology consisting of drug and carrier, therefore named binary composites.

While, the present invention allows to effectively administer poorly bioavailable drugs, the known binary Biorise composite do not even have ability to control and trigger the release of the activated drug according to external stimuli (i.e. pH changes); also in this case further manufacturing steps (i.e. film coating) should be applied.

### BRIEF DESCRIPTION OF THE DRAWINGS

The invention will be now described in relation to the following Figures, wherein:

FIG. 1 The biopharmaceutical classification system (BCS)

FIG. 2. Modifications applied to standard USP II dissolution apparatus

FIG. 3. DSC traces of 20% drug load reference binary composite (REFERENCE 1) and of 20% (1:3:1) ternary composite containing vinylpyrrolidone vinyl acetate copolymer (SAMPLE 1)

FIG. 4. DSC traces of 20% drug load reference binary composite (REFERENCE 1) and of 20% (1:3:1) ternary composite containing polyethyleneglycol-caprolactame-vinylpyrrolidone copolymer copolymer (SAMPLE 3)

FIG. 5. DSC traces of 20% drug load reference binary composite (REFERENCE 1) and of 20% (1:3:1) ternary composite containing dimethylaminoethyl methacrylate-butylmethacrylate-methylmethacrylate copolymer (SAMPLE 5)

FIG. 6. DSC traces of 20% drug load reference binary composite (REFERENCE 1) and of 20% (1:3:1) ternary composite containing polyvinylpyrrolidone (SAMPLE 2)

FIG. 7. DSC traces of 20% drug load reference binary composite (REFERENCE 1) and of 20% (1:3:1) ternary composite containing polyoxyethylene-polyoxypropylene copolymer (SAMPLE 4)

FIG. 8. Comparison of XRPD traces of ternary composite containing polyoxyethylene polyoxypropylene copolymer (SAMPLE 4) and of physical blend of its components

FIG. 9. DSC traces of 25% drug load reference binary composite (REFERENCE 3) and of 25% (1:2:1) ternary composite containing vinylpyrrolidone-vinyl acetate copolymer (SAMPLE 6)

FIG. 10. DSC traces of 20% reference binary composite (REFERENCE 2) and 20% ternary composite containing vinylpyrrolidone-vinyl acetate copolymer (SAMPLE 7)

FIG. 11. DSC traces of 20% binary composite (REFERENCE 2), recorded on instrument and with procedure used for QDSC

FIG. 12. Reversible and Irreversible events DSC traces of 20% ternary composite containing vinylpyrrolidone-vinyl acetate copolymer (SAMPLE 7)

FIG. 13. DSC traces of 20% ternary composite containing vinylpyrrolidone-vinyl acetate copolymer (SAMPLE 7), recorded on instrument and with procedure used for QDSC

FIG. 14. XRPD traces of 20% binary composite (REFERENCE 2) and of fenofibrate-cross-linked polyvinylpyrrolidone physical blend

FIG. 15. Fenofibrate crystalline domains size distribution of binary composite 1:4 sample (REFERENCE 2)

FIG. 16. DSC traces of ternary composite 1:18:1 (SAMPLE 9) and binary composite 1:19 (REFERENCE 5)

FIG. 17. DSC traces of ternary composite 1.8:1 (SAMPLE 8) and binary composite 1:9 (REFERENCE 4)

FIG. 18. Solubilization kinetic profiles of fenofibrate in physical blend; oversaturation factor 150× in pH 1.2 medium

FIG. 19. Details of solubilization kinetic profiles presented in FIG. 18

FIG. 20. Solubilization kinetic profiles of composites; oversaturation factor 150× in pH 1.2 medium

FIG. 21. Solubilization kinetic profiles of ternary (SAMPLE 7) and binary (REFERENCE 2) fenofibrate composites (20% drug load); manual method; oversaturation factor 75× in pH 1.2 medium

FIG. 22. Solubilization kinetic profiles of ternary (SAMPLE 11) and binary (REFERENCE 7) fenofibrate composites (20% drug load); lab scale method, oversaturation factor 75× in pH 1.2 medium

FIG. 23. Two stages solubilization kinetic experiment on fenofibrate ternary composite (20% w/w drug load) containing dimethylaminoethyl methacrylate-butylmethacrylate-methylmethacrylate copolymer; first stage (0-600 seconds) at pH 6.8, second stage (601-1200 seconds) at pH 1.2. pH shift obtained by addition of phosphoric acid to the pH 6.8 buffer; oversaturation factor 75×

FIG. 24. Solubilization kinetic profile of binary and ternary composites with 10% drug load (1:9 and 1:8:1); oversaturation factor 75× in pH 1.2 medium

FIG. 25. Solubilization kinetic profiles of ternary and binary fenofibrate composites; 20% and 25% drug load; oversaturation factor 150× in pH 1.2 medium

FIG. 26. Solubilization kinetic profiles of binary composites at 20% and 25% drug load

FIG. 27. Solubilization kinetic profile of binary and ternary composites with 5% drug load (1:19 and 1:18:1); oversaturation factor 40× in pH 1.2 medium

FIG. 28. Solubilization kinetic profiles of ternary composites containing dimethylaminoethyl methacrylate-butylmethacrylate-methylmethacrylate copolymer or vinylpyrrolidone-vinyl acetate copolymer; oversaturation factor 75× in pH 1.2 medium

FIG. 29. DSC trace of the pure nifedipine

FIG. 30. DSC traces of nifedipine-vinylpyrrolidone-vinyl acetate copolymer, DSC traces of nifedipine-dimethylaminoethyl methacrylate-butylmethacrylate-methylmethacrylate copolymer 1:1 physical blends, DSC trace of nifedipine

FIG. 31. DSC traces of 20% drug load binary composite (REFERENCE 8)

FIG. 32. Solubilization kinetic profiles of nifedipine physical blends; SK of nifedipine; scattering wavelength is 600 nm

FIG. 33. Solubilization kinetic profiles of nifedipine 20% binary composite (REFERENCE 8) and two nifedipine 20% ternary composites (SAMPLE 14, SAMPLE 15); oversaturation factor 25× in pH 1.2 buffer; scattering wavelength 500 nm

FIG. 34. Solubilization kinetic profiles of SAMPLE 16, REFERENCE 9 and REFERENCE 10

FIG. 35. Drying curve for SAMPLES 17a, b, c

FIG. 36. Solubilization kinetic profiles of SAMPLES 17a, b, c

FIG. 37. Drying curve for SAMPLES 12a, b, c

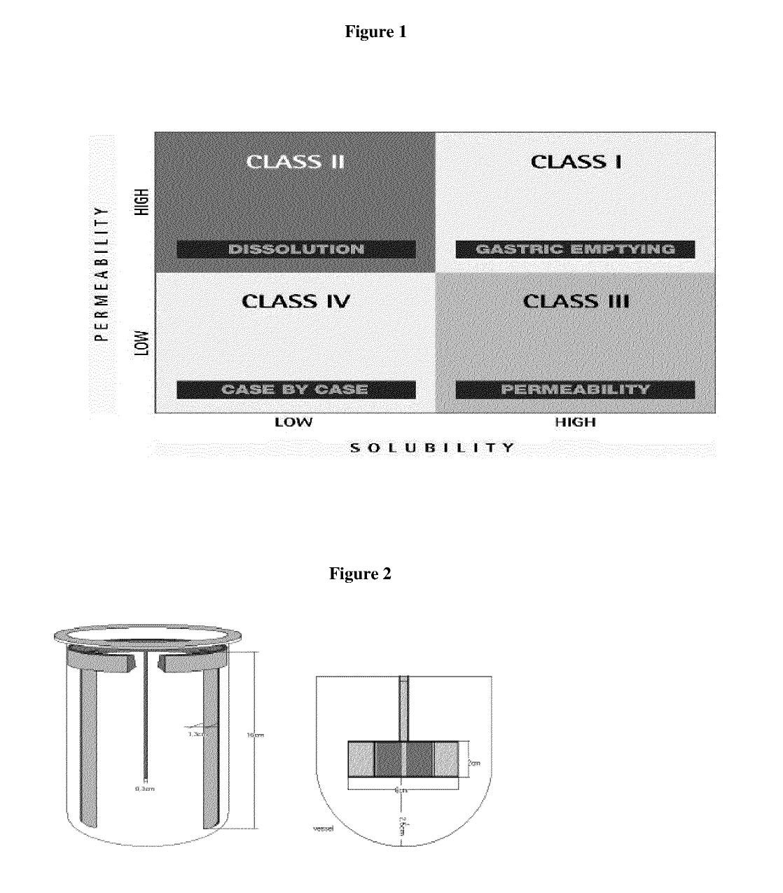

### DETAILED DESCRIPTION OF THE INVENTION

The present invention is directed to a composite comprising at least one poorly soluble drug, at least one polymeric carrier and at least one not chemically cross-linked polymer, which is both soluble in water and organic solvent. The disclosed composites are also defined as ternary composites.

With regards to the drugs, the invention is applicable to poorly soluble drugs; the drug fall into one or more of the following classes of drugs: abortifacient/interceptive agents; ace-inhibitors; alpha- and beta-adrenergenic agonists; alpha- and beta-adrenergic blockers; adrenocortical steroids and suppressants; adrenocorticotropic hormones; alcohol deterrents; aldose reductase inhibitors; aldosterone antagonists; ampa receptor antagonists; anabolics; angiotension II receptors; anorexics; antacids; anthelmintics; antiacne agents; antiallergics; antialopecia agents; antiamebics; antiandrogens; antianginals; antiarrhythmics; antiarthritics/antirheumatics; antibiotics (natural and synthetic); anticoagulants; anticonvulsants; antidepressants; antidiabetics; antidiarrheal; antidiuretics; antiemetics; antiglaucoma agents; antigout agents; antihistaminics; antihyperlipoproteinemics; antihyperparathyroids; antiper-phosphatemics; antihypertensives; antiperthyroids; antihypotensives; antihypothyroid agents; antiinflammatories (non-steroidal and steroidal); antimalarials; antimigraine agents; anti-muscarinics; antineoplastics; antiobesity agents; antiobsessional agents; antiosteoporotic agents; antiparkinsonian agents; antiprotozoal agents; antipruritics; antisporiatics; antipsychotics; antipyretics; antispasmodics; antithrombotics; antitussives; antiulceratives; antivirals; anxiolytics; calcium channel blockers; calcium regulators; carbonic anhydrase inhibitors; cardioprotectives; cardiotonics; choleretic agents; cholinergics; cholinesterase inhibitors; central nervous system stimulants; contraceptives; decongestants; diuretics; dopamine receptor agonists and antagonists; expectorants; fibrinogen receptor antagonist; glucocorticoids; hematinics; immunomodulators; immunosuppressants; monoamine oxidase inhibitors; mucolytics; muscle relaxants; mydriatics; narcotic antagonists; neuromuscular blocking agents; neuroprotectives; nootropics; prolactin inhibitors; reverse transcriptase inhibitors; sedatives/hypnotics; serotonin receptor agonists and antagonists; serotonin uptake inhibitors; steroids, thrombolytics; vasodilators; and vitamins. Examples of poorly soluble drugs falling within the above groups are: fexofenadine, nifedipine, griseofulvin, indomethacin, diacerein, megestrol acetate, estradiol, progesterone, medroxyprogesterone acetate, nicergoline, clonidine, etoposide, lorazepam, temazepam, digoxin, glibenclamide ketoprofen, indobufen, ibuprofen, nimesulide, diclofenac, naproxene, acemethacine, raloxifene, paroxetine, glimepiride, anagrelide, modafanil, paroxetine, cabergoline, replaginide, glipizide, benzodiazapines, clofibrate, chlorpheniramine, digoxine, diphen-hydramine, egrotamine, estradiol, fenofibrate, griseofulvin, hydrochlothizide, hydrocortisone, isosorbide, medrogeston, oxyphenbutazone, prednisolone, prednisone, polythiazide, progesterone, spirono-lactone, tolbutamide, phenacetin, phenyloin, digitoxin, nilvadipine, diazepam, griseofulvin and chloramphenicol.

The composite has drug load (amount of drug) comprised from about 2 to about 65% weight of the drug with respect to the weight of the composite; preferably from about 3 and 48% w/w; preferably from 5 to about 45% w/w even more preferably from about 5 to about 34% w/w; it may be about 2%, about 3%, about 5%, about 10%, about 15%, about 20%, about 25%, about 33.3%, about 34%, about 40%, about 45%, about 48%, about 65% w/w.

Drug/polymeric carrier weight ratio ranges from 1:0.5 to 1:50 w/w, preferably from 1:1 to 1:18 w/w; specific examples of this ratio are 1:2, 1:3, 1:8, 1:18 w/w.

The weight ratio between the drug and the water and organic solvent soluble polymer may range from 1:0.1 to 1:10 w/w, preferably from 1:0.2 to 1:5 w/w, preferably it may be 1:0.5, 1:1 or 1:2 w/w.

The preferred amount of the three components by weight of the composite is 1 part of drug, 1-18 (preferably 2-3) parts of polymeric carrier, 0.5-1.5 (preferably 1) parts of water and organic solvent soluble polymer.

The carrier is a cross-linked polymer, which is insoluble but swellable in aqueous media and in organic solvents, it may be a mixture of one or more such polymers. Examples of suitable polymers are: cross-linked polyvinylpyrrolidone (crospovidone), cross-linked sodium carboxymethylcellulose, cross-linked cyclodextrins, cross-linked dextran, cross-linked starch (i.e. sodium starch glycolate), cross-linked methylcellulose. Particular interesting is the cross-linked polyvinyl pyrrolidone.

The not chemically cross-linked polymer, which is both soluble in water and organic solvent, is a polymer which combines dual solubility, that is the polymer is soluble not only in water but also in organic solvent. his polymer is soluble in organic solvent and in water at all pH values, i.e. in water having a pH comprised from 1 to 14, preferably pH from 1 to 7.5. This polymer may have a pH independent or dependent solubility: this means that in a first embodiment the polymer is soluble at all pH values (pH independent) and in a second embodiment it is soluble at specific pH value in all pH range (pH dependent). The polymer with pH dependent solubility is soluble at pH equal or lower than 5 or it is soluble at pH equal or higher than pH 5, or equal or higher than pH 5.5, or equal or higher than pH 6 or equal or higher than pH 6.5 or equal or higher than pH 6.8. The water in which the polymer (both the pH dependent and the pH independent) is soluble, may comprise buffers or salts providing the different pH and/or ionic strength to it, it includes also physiological solutions (such as gastric fluid, intestinal fluid). The term “not chemically cross-linked polymer”, excludes both covalently and not covalently cross-linked polymers. The not chemically cross-linked polymer which is both soluble in water and organic solvent used in the present invention is hereafter called “soluble polymer” or “water and organic solvent soluble polymer”.

Non-limiting examples of the not chemically cross-linked polymer which is both soluble in water and organic solvent are: cellulose derivatives such as: hydroxypropylcellulose, hydroxyethylcellulose, hydroxypropylmethylcellulose, hydroxypropylmethycellulose acetate succinate, cellulose acetate trimellitate, etc; acrylic and methacrylic polymers and their copolymers such as: methacrylic acid-methylmethacrylate copolymer, polyaminoalkyl methacrylate-methacrylic esters copolymer, dimethylaminoethyl methacrylate-butylmethacrylate-methylmethacrylate copolymer (Eudragit® E); linear polyvinylpyrrolidone (povidone or PVP, i.e. Kollidon® K30, BASF, Polyplasdone®, ISP), vinylpyrrolidone-vinyl acetate copolymer (copovidone, i.e. Kollidon® VA64, BASF), methylvinylether-maleic acid copolymer, polyethyleneglycol-caprolactame-vinylpyrrolidone copolymer (Soluplus®), polyoxyethylene-polyoxypropylene (Poloxamer, i.e.—Lutrol® F68, BASF). Among the above listed polymers, the polymers having pH dependent solubility that may dissolve in water at pH from 1 to 5 or pH from 5 to 14, may be selected from the group consisting of dimethylaminoethyl methacrylate-butylmethacrylate-methylmethacrylate copolymer (Eudragit® E) (soluble at pH equal or lower than 5), methacrylic acid-methylmethacrylate copolymer (soluble at pH equal or higher than pH 6 or equal or higher than pH 6.5), hydroxypropylmethycellulose acetatesuccinate (soluble at pH equal or higher than pH 5.5 or equal or higher than pH 6 or equal or higher than pH 6.5 or equal or higher than pH 6.8), cellulose acetate trimellitate (soluble at pH equal or higher than pH 5). Preferred polymers for the composite of the present inventions are: vinylpyrrolidone-vinyl acetate copolymer polyvinylpyrrolidone, dimethylaminoethyl methacrylate-butylmethacrylate-methylmethacrylate copolymer (i.e. Eudragit® E, Evonik).

Further object of the present invention is also the process for the preparation of the ternary composite herein disclosed; it comprises the following steps:

1) Dissolving at least one poorly water soluble drug in a process solvent or process solvent mixture;
2) Dissolving at least one water and organic solvent soluble polymer into the drug solution of step 1);
3) Swelling at least one the polymeric carrier with the solution prepared in step 2) thus obtaining a swollen composite;
4) Removing the process solvent from the swollen composite of step 3).

With the term process solvent or process solvent mixture is herein intended a solvent or solvent mixture suitable to be used in the process of the invention.

Alternatively, steps 1) and 2) can be performed by dissolving simultaneously at least one poorly soluble drug and at least one water and organic solvent soluble polymer. In other word, the drug and the polymer are either added in the same vessel, the solvent or solvent mixture is poured thereon and the dissolution of the components is obtained, preferably under stirring, or the drug and the not chemically cross-linked polymer are each separately solubilized in the process solvent and the two solutions are then mixed together; the process solvent may be the same or may be different.

In this alternative embodiment the process consists of following steps:

1-2bis) Dissolving at least one poorly water soluble drug and at least one not cross-linked polymer, which is both water and organic solvent soluble, in process solvent or process solvent mixture;
3) Swelling at least one polymeric carrier with the solution prepared in step 1-2bis), thus obtaining a swollen composite;
4) Removing the process solvent from the swollen composite of step 3).

The above process can also be further slightly modified to obtain the ternary composites of the invention; the alternative method comprises the same steps as above but applied in a different order; that is the water organic solvent soluble polymer is added after the swelling of the polymeric carrier; this modified process comprises the above steps applied in the following order:

a1) Dissolving at least one drug in an process solvent or solvent mixture;
a2) Swelling the polymeric carrier with the solution prepared in step a1) thus obtaining a swollen composite;
a3) Removing the process solvent from the swollen composite of step a2), thus obtaining a binary composite (drug and polymeric carrier);
a4) Dissolving at least one water and organic solvent soluble polymer in a process solvent or solvent mixture; to
a5) Swelling the binary composite of step a3) with the solution of step a4) thus obtaining a ternary swollen composite;
a6) Removing the process solvent from the ternary swollen composite of step a5), thus obtaining the ternary composite.

For the preparation of the solutions of steps 1) and 2) (or corresponding 1-2bis) step or a1) and a4) steps), the weight ratio of the organic solvent to the carrier is chosen on the basis of the carrier swelling capacity, that is the maximum amount of solvent that the carrier can absorb by unit weight without having free liquid outside the solid particles. For example, in case of cross-povidone and acetone this value ranges from 2.0 to 2.5 g of pure solvent by g of carrier. The presence of the drug and/or polymer may modify the quantity of solution that can be absorbed by the carrier, usually decreasing it if compared to the pure solvent.

The final concentration of the drug/polymer solution results from the amount of solvent required by the carrier and by the drug and water and organic solvent soluble polymer ratios to the carrier. The solvent or solvents mixtures suitable for use in the process according to the invention are all those which are able to swell the polymeric carrier or to be absorbed by the carrier polymer and to dissolve the drug and the water and organic solvent soluble polymer selected. Examples of solvents are methanol, ethanol, higher alcohols, acetone, chlorinated solvents, formamide, dimethylformamide, fluorinated hydrocarbons and others or mixture thereof. Preferred solvents are acetone, dichloromethane, dimethylformamide.

The swollen composite of the invention is obtained by the swelling of step 3) (or corresponding a6) step), which comprises the contacting of the solution of step 2) (or corresponding a4) step), with the polymeric carrier and the homogeneously distribution (homogenization) of the solution of step 2) (or corresponding a4) step) within this mass. During this step it is important to reduce possible solvent loss from the mass. The homogeneous distribution can be obtained in different ways depending on process scale and equipment availability. The homogeneous distribution of the solution within the material can be achieved by mixing. When the equipment has a container which is not tightly closed (such as for manual preparation process of small amount of composite) then the homogeneous distribution is achieved by exposing the material for a defined period of time (preferably ranging from 0.5 to 24 hours) preferably at room temperature to process solvent vapors; in this way homogeneous distribution of the solution within the material is reached with minimal loss of solvent. To prevent solvent loss during this step, the use of equipment with tightly closed process container is preferred.

The process solvent removal step (step 4, or corresponding a3) and a7) steps) is conducted to achieve a suitable residual level of solvent in the final composite. Acceptable residual solvent level depends on the solvent and is herein defined as the highest limit provided by the ICH guidelines. As an example, the ICH guidelines limit for class 3 solvent (such as acetone) is 5,000 ppm. For dichloromethane and dimethylformamide the ICH limit is respectively 600 ppm and 880 ppm being both class 2 solvents. This drying step is performed under controlled conditions of time duration; in fact, it is carried out for a short period of time, since this parameter may affect the final structure, characteristics and performance of the composites, depending on their qualitative and/or quantitative composition. In particular, it is important that the final desired residual solvent amount is achieved in the shortest period of time as possible. The temperature and exposure to humidity applied during this step may also be important parameters to be controlled.

The removal of process solvent is carried out for a short period of time. This period of time is preferably equal or shorter than about 410, or about 400, or about 360, or 240, or about 180, or about 120 or about 15 minutes. This time duration may be affected by the amount of swollen composite to be dried, its solvent content and the equipment used.

The process temperature during the fast drying step is above room temperature, from about 30° C. to about 100° C. depending on the process solvent used and vacuum application. The temperature is preferably from about 35 to about 60° C., or from about 40 to about 55° C., or from about 45 to about 50° C.; the temperature may be about 30° C., about 35° C., about 40° C., about 45° C., about 43° C., about 49° C., about 50° C., about 55° C., about 60° C.

The removal of process solvent is preferably carried out for a period of time which equal or shorter than about 410 and at temperature from 30 to 100° C.; or for a period of time which is equal or shorter than about 360 minutes and at temperature from 30 to 100° C.; or for a period of time which is equal or shorter than about 240 minutes and at temperature from 30 to 100° C.; or for a period of time which is equal or shorter than about 120 minutes and at temperature from 30 to 100° C. Or the removal of process solvent is preferably carried out for a period of time which equal or shorter than about 410 and at temperature from 35 to 60° C.; or for a period of time which is equal or shorter than about 360 minutes and at temperature from 35 to 60° C.; or for a period of time which is equal or shorter than about 240 minutes and at temperature from 35 to 60° C.; or for a period of time which is equal or shorter than about 120 minutes and at temperature from 35 to 60° C. Or the removal of process solvent is preferably carried out for a period of time which equal or shorter than about 410 and at temperature from 40 to 55° C.; or for a period of time which is equal or shorter than about 360 minutes and at temperature from 40 to 55° C.; or for a period of time which is equal or shorter than about 240 minutes and at temperature from 40 to 55° C.; or for a period of time which is equal or shorter than about 120 minutes and at temperature from 40 to 55° C. The fast solvent removal step (drying step) may include a quick pre-drying step. This pre-drying step is in particular useful when the drying step is performed in equipment other than that where the swelling step is carried out. This occurs for example with the manual process or with the process carried out in mixer/granulator, which do not have heating capacity or has a limited drying efficiency (apparatus not suitable for “one pot” process). In case of the manual process, the homogenized material may be left at room temperature, possibly under vacuum, before being transferred into the heated dryer for the drying process (such as a vacuum oven), in this way fast partial solvent evaporation is achieved and crust formation is avoided (crust may slow down the subsequent solvent removal). In case the swelling and the homogenization are conducted in a mixer/granulator, the partial fast removal of solvent (pre-drying) leads to a wet powder easier to be transferred into the dryer for step 4) completion than the viscous-creamy swollen product.

Also this “pre-drying” step, should be fast, that means its duration should shorter than about 90, or about 85, or about 80, or about 40, or about 35 minutes. The temperature can be room temperature or above; it can be from about 20° C. to about 100° C. depending on the process solvent used and vacuum application. The temperature is preferably from about 20 to about 60° C., or from about 25 to about 55° C.; the temperature may be about 20° C., about 25° C., about 55° C., about 60° C.

When vacuum is applied during the drying step, the drying duration is significantly reduced and lower temperature may be applied. Vacuum pump or centralized vacuum system can be used to reduce pressure inside dryers; lower the residual pressure into the drying chamber, faster the solvent removal. For examples, with the equipments used in the experimental parts, a residual pressure value from about 0.30 to about 0.40 bar, or from about 0.30 to about 0.20 bar, or below about 0.20 bar is reached.

When dryer with no vacuum capacity is used (such as with fluid bed dryer) it is preferred to use low humidity process gas, that means water content in the range of about 4.0-5.0 g water/Kg of gas or below. This is important to reduce risk of chemical or physical instability of the drug into the composite.

The preferred solvent in the process of the invention is acetone and all the above listed ranges and values related to solvent removal rate, time duration, process temperature, residual pressure, humidity of gas during both the pre-drying and the drying apply also to this specific preferred solvent.

This removal of process solvent can be conducted in different ways, depending mostly on the scale applied and on the equipment availability. All types of direct heating dryers (heat transfer mainly by thermal conduction), indirect heating dryers (heat transfer mainly by thermal convection) and radiant dryers (heat transfer mainly by electromagnetic and dielectric radiation) can be used in the present invention. Preferred dryers operates under vacuum because they allow significant reduction of drying time and temperature; moreover in this type of equipment contact with moisture is limited or even avoided, with possible benefit for the composite physical and chemical stability.

Examples of dryers that can be used in step 4) (or corresponding a3) and a6) steps), of the process of the invention are: jacketed low shear mixer/granulator/dryer (“one pot”, indirect heating equipment), vacuum oven (indirect heating equipment), fluid bed dryer (direct heating equipment), microwave assisted dryer (dielectric heating equipment), microwave assisted high shear mixer/granulator/dryer (“one pot”, dielectric heating equipment), infrared assisted dryer (electromagnetic heating equipment). Equipments combining mixing and vacuum drying capacity are very interesting, because they allow combination of steps 3) and 4) into a single machine (“one pot process”). Other equipments that may be used under the operative conditions described above can also be used.

More details about the different process steps with regards to parameters, operative conditions, amounts are given in the experimental part.

It is understood that all embodiments (including the process parameter values) described herein can naturally be combined with one another.

Without being bound to any theory, it is believed that the outstanding properties of the composite of the invention are mainly achieved by the combination of the described three components. The condition of fast solvent removal from the swollen composite is also an important feature for the optimization of the composite manufacturing and for the composite itself.

The present invention discloses also pharmaceutical compositions and dosage forms comprising the composite of this invention and further pharmaceutically acceptable excipients. Excipients for use in the compositions or dosage forms of the present invention include fillers, diluents, glidants, disintegrants, superdisintegrants, binders, lubricants, etc. Other pharmaceutically acceptable excipients include acidifying agents, alkalizing agents, preservatives, antioxidants, buffering agents, chelating agents, coloring agents, complexing agents, emulsifying and/or solubilizing agents, flavors and perfumes, humectants, sweetening agents, wetting agents etc.

Examples of suitable fillers, diluents and/or binders include, but are not limited to, lactose (e.g. spray-dried lactose, α-lactose, (β-lactose, Tabletose®, various grades of Pharmatose®, Microtose® or Fast-Floc®), microcrystalline cellulose (e.g. Avicel® PH101, Avicel® PH102, Ceolus® KG-802, Ceolus® KG-1000, Proslv® SMCC 50 or SMCC90, various grades of Elcema®, Vivacel®, Ming Tai® or Solka-Floc®), hydroxypropylcellulose, L-hydroxypropylcellulose (low substituted), hydroxypropyl methylcellulose (HPMC) (e.g. Methocel® E, F and K, Metolose® SH of Shin-Etsu, Ltd, such as, e.g., the 4,000 cps grades of Methocel® E and Metolose® 60 SH, the 4,000 cps grades of Methocel® F and Metolose® 65 SH, the 4,000, 15,000 and 100,000 cps grades of Methocel® K; and the 4,000, 15,000, 39,000 and 100,000 grades of Metolose® 90 SH), methylcellulose polymers (such as, e.g., Methocel® A, Methocel® A4C, Methocel® A15C, Methocel® A4M), hydroxyethylcellulose, sodium carboxy-methylcellulose, carboxymethylhydroxyethylcellulose and other cellulose derivatives, sucrose, xanthan gum, cyclodextrin, agarose, sorbitol, mannitol, dextrins, maltodextrins, starches or modified starches (including potato starch, maize starch and rice starch), calcium phosphate (e.g. basic calcium phosphate, calcium hydrogen phosphate, dicalcium phosphate hydrate), calcium sulfate, calcium carbonate, sodium alginate, collagen etc. or combinations thereof.

Crospovidone may also be added as superdisintegrant.

Specific examples of diluents include, e.g. calcium carbonate, dibasic calcium phosphate, tribasic calcium phosphate, calcium sulfate, microcrystalline cellulose, powdered cellulose, dextrans, dextrin, dextrose, fructose, kaolin, lactose, mannitol, sorbitol, starch, pregelatinized starch, sucrose, xanthan gum, cyclodextrin, and combinations thereof.

Specific examples of glidants and lubricants include, e.g., silicon dioxide, stearic acid, magnesium stearate, calcium stearate or other metallic stearates, talc, waxes and glycerides, light mineral oil, PEG, glyceryl behenate, colloidal silica, hydrogenated vegetable oils, corn starch, sodium stearyl fumarate, polyethylene glycols, alkyl sulfates, sodium benzoate, sodium acetate etc.

Other excipients include, e.g., flavoring agents, coloring agents, taste-masking agents, pH-adjusting agents, buffering agents, preservatives, stabilizing agents, anti-oxidants, wetting agents, humidity-adjusting agents, surface-active agents, suspending agents, surfactants, absorption enhancing agents, agents for modified release etc.

Non-limiting examples of flavoring agents include, e.g., cherry, orange, banana, strawberry or other acceptable fruit flavors, or mixtures of cherry, orange, and other acceptable fruit flavors, at up to, for instance, about 3% based on the tablet weight. In addition, the compositions of the present invention is can also include one or more sweeteners such as aspartame, sucralose, or other pharmaceutically acceptable sweeteners, or mixtures of such sweeteners, at up to about 2% by weight, based on the tablet weight. Furthermore, the compositions of the present invention can include one or more FD&C colorants at up to, for instance, 0.5% by weight, based on the tablet weight.

Antioxidants include, e.g., ascorbic acid, ascorbyl palmitate, butylated hydroxyanisole, butylated hydroxytoluene, hypophosphorous acid, monothioglycerol, potassium metabisulfite, propyl gallate, sodium formaldehyde sulfoxylate, sodium metabisulfite, sodium thiosulfate, sulfur dioxide, tocopherol, tocopherol acetate, tocopherol hemisuccinate, TPGS or other tocopherol derivatives, etc.

The composites of the invention may be formulated into a variety of final dosage forms including tablets (e.g. orally disintegrating chewable, dispersible, fast dissolving, effervescent), hard gelatin capsules. Sprinkle, suspensions, sachets for permanent or extemporaneous suspensions, and sachets for direct administration in the mouth are also examples of dosage forms. In particular with some composite of the present invention it is possible to strongly reduce the drug release in aqueous media at pH>5; in this case the release starts as soon as the medium pH is lowered to 1-2 (gastric fluid) and the amount of dissolved drug and solubilization kinetic (SK) profile are equivalent to those observed for the same composite in acidic medium. Differently, the binary composite samples suspended in water before solubilization kinetic test show poorer performance than similar samples tested as solid powder.

Several advantages of the present invention will become clear from the reading of the experimental part, such as for example the higher content of amorphous and/or nano-crystalline drug which is achieved with the composite of present invention with respect to the known binary Biorise composite. Moreover, these composite are highly stable upon storage.

### Experimental Part

The following experiments are presented as non limiting examples of the invention. In all the cases the ternary composites are defined as “SAMPLE”, the known binary Biorise composites are defined as “REFERENCE”.

### 1) Materials

1.1. Drugs: fenofibrate (FF), nifedipine (ND) and nimesulide (NM) are used herein as representative poorly soluble drugs for the composites of the invention. Fenofibrate is poorly water soluble (from 0.3 to 0.8 μg/ml) with pH independent solubility. Nifedipine is also poorly water soluble, even if its pH independent equilibrium solubility, 5 μg/ml, is higher than that of fenofibrate. Nimesulide is also poorly water soluble, with pH dependent solubility ranging from about 20 μg/ml at pH 2.5 to about 90 μg/ml at pH 10.

1.2. Organic solvent: acetone is one of the preferred solvent for the SIA process; it has low boiling point, good solvent capacity for many drugs, minor safety concern for human use and for ambient pollution.

1.3. Carrier: cross-linked polyvinylpyrrolidone (CPVP) (Kollidon® CL-M) is chosen as preferred carrier.

1.4. Water and organic solvent soluble polymers: pharmaceutically acceptable polymers used herein are vinylpyrrolidone-vinyl acetate copolymer (Kollidon® VA64), polyvinylpyrrolidone (Kollidon® K30), polyoxyethylene polyoxypropylene copolymer (Lutrol® F68), polyethyleneglycol-caprolactame-vinylpyrrolidone copolymer (Soluplus®); dimethylaminoethyl methacrylate-butylmethacrylate-methylmethacrylate copolymer (Eudragit® E) is also tested. Their properties are described in Table 1.

TABLE 1
Properties of the water and organic solvent soluble polymers

| | |
|---|---|
| Surfactant | Solubility in process solvent (acetone) |

| capacity | Poor | High |
|---|---|---|
| No | Polyvinylpyrrolidone | Vinylpyrrolidone- |
| | (Kollidon ® K30) | vinyl acetate |
| | | (Kollidon ® VA64) |
| Yes | Polyoxyethylene- | Polyethyleneglycol- |
| | polyoxypropylene | caprolactame- |
| | copolymer | vinylpyrrolidone |
| | (Lutrol ® F68) | copolymer |
| | | (Soluplus ®) |

### 2) Composites Characterization Methods

### 2.1 Loss on Drying Test.

Sample size is about 1.5-2.5 g. Thermobalance Mettler-Toledo HR73 is used and test is conducted at heating temperature of 100° C. reached with fast ramp applying, automatic stop at constant weight with sensitivity level 3. Test result is expressed as percentage loss of the starting (wet) weight and it is used for a rough estimation of the amount of organic solvent into the composites during process steps.

### 2.2 Differential Scanning Calorimetry (DSC).

The presence of drug in crystalline form is qualitatively assessed using differential scanning calorimetry, seeking for the drug melting endothermal peak. Quantification of the amount of crystalline fenofibrate in composite is performed using a drug specific Quantitative DSC (QDSC) method based on measuring the melting enthalpy value into composite. DSC cannot be used when the heating applied to samples induces interactions between drug and excipients. Analysis of DSC traces of drug/excipients binary physical blends is used to point out the interacting materials. DSC scans are acquired on two instruments with different procedures:

Procedure 1) is applied for the preliminary qualitative evaluation of solid phases. It is conducted on DSC6 differential scanning calorimeter (Perkin Elmer, USA). An amount of composite corresponding to about 1.0-1.5 mg of drug is accurately weighed into aluminum pan; pan lid is fixed in position and the analysis is conducted under nitrogen flow (20 ml/min) at scanning rate of 10° C./min from 25° C. to final temperature selected according to the target drug: 120° C. for fenofibrate and 200° C. for nifedipine. This method is also applied for the analysis of physical blends of target drug and composite components useful to evaluate interactions.

Procedure 2) is applied for the quantitative scans (QDSC). It is conducted on a power-compensated differential scanning calorimeter Pyris-1 (Perkin Elmer, USA). About 5-6 mg of composite are accurately weighed into aluminum DSC pan, pan lid is fixed in position and the analysis is conducted under nitrogen flow (20 ml/min) at scanning rate of 10° C./min from −20° C. to final temperature selected depending on the drug: 120° C. for fenofibrate and 200° C. for nifedipine.

### 2.3 Thermal-Gravimetric Analysis (TGA).

Thermogravimetric analysis (Pyris 1, Perkin Elmer) are conducted on Pyris 1 instrument; 8-9 mg of composite samples are tested under a nitrogen stream of 35 ml/min at scanning rate of 10° C. min-1 from 18° C. to 150° C. (only for fenofibrate).

2.4 X-Ray Powder Diffraction (XRPD) measurements are performed on a Philips X'Pert PRO diffractometer (Bragg-Brentano geometry). CuK lambda radiation (lambda=1.541 Angstrom), generated by a sealed X-Ray tube (40 kV×40 mA), and a real time multiple strip detector (X' Celerator, Philips). Samples are prepared in a back-loading sample holder and analyzed using Spinner module. Angular range is 5°-40°.

2.5 Assay of fenofibrate is performed by quantitative HPLC (Agilent 1100, DAD detector module equipped with automatic injector with injection volume 25 microlitre Waters Symmetry C18 column (150×4.6 mm; particle size 3.5 microns); mobile phase is a mixture of acetonitrile/water in the ratio 70/30 v/v containing 0.1% of trifluoroacetic acid. The following settings are used: flow rate equal to 1.0 ml/min; run time 13 minutes and the column temperature is 25° C. Fenofibrate retention time is 10.5 minutes. The eluents are monitored at 280 nm. The assay is determined by comparing the peak area of the sample solution with that of the standard solution.

2.6 Solubilization kinetic test (SK) has been developed to investigate and highlight the effect of physical-chemical modifications (i.e. solid state change) on the solubility of poorly soluble drugs. It is conducted using an USP type II apparatus (Sotax AT6) modified by substituting the standard paddle with a six blades impeller (FIG. 2) operated during the test at high speed (i.e. 150 rpm) to create turbulent hydrodynamic into the medium contained in the 1000 ml vessel. This helps the powder dispersion into the medium, making negligible the effect of composite wetting/dispersion on the drug release into solution. The samples (composites, composite aqueous suspension or drug/excipients physical blends) are tested in 500 mL of aqueous buffer kept at 37° C. A quantity of sample corresponding to a fixed amount of target drug in large excess (at least 10-15 times) to its equilibrium solubility is weighed for each test and added into the vessel under stirring. The amount of dissolved drug is continuously determined using a spectrophotometer MCS 551-UV equipped with an optical fiber with 10 mm or 2 mm path length respectively in case of fenofibrate and nifedipine samples. The net absorbance at the analytical wavelength is used for quantification of the target drug concentration against a reference standard. Being SK a dispersion method, the net absorbance of the target drug is estimated subtracting from the absorbance at analytical wavelength the value measured at wavelength far from any drug absorbance (scattering wavelength), to take into account the fraction of light scattered by solid particles suspended into the SK test medium.

Two types of test are conducted for the characterization of the composites:

1) Single step test: pH 1.2 aqueous buffer is used; the drug concentration is continuously measured for ten minutes.
2) Two steps (or two stages) test: the testing material is dispersed under stirring into 500 ml of phosphate buffer at pH 6.8, the dissolved drug concentration is continuously measured for ten minutes, then 13.5 ml of orthophosphoric acid (85%) are added to reduce pH at about 1.2 and the dissolved drug concentration is measured for further ten minutes before closing the test.

### 2.7 Solvent Removal Curve.

During drying, samples are taken at different times and the measured amount of solvent is plotted against process time. The amount of solvent is measured by loss on drying (LoD) test (in process estimation) or by gas-chromatography (precise quantification).

### 3. Processes for the Preparation of Composites

### 3.1 Manual Method

Batch size of composite is 10 g, unless otherwise specified; process details applied for the batches manufactured with this method are reported in Table 2 and Table 3 with the relevant samples codes. The required amount of target drug is accurately weighed and dissolved under magnetic stirring into the appropriate quantity of process solvent (acetone unless otherwise specified). Then, the required amount of water and organic solvent soluble polymer is accurately weighed and added under stirring to the solution of drug in acetone. Stirring is continued until polymer complete dissolution or homogeneous dispersion. The quantity of acetone is, unless otherwise specified, about 2.3 time the weight of cross-linked polyvinylpyrrolidone, according to the “swelling index” of this carrier polymer in the selected organic solvent. The organic solution is slowly poured on the required amount of cross-linked polyvinylpyrrolidone previously weighed into a ceramic mortar of suitable size. Liquid and solid are mixed using a small metallic spatula, to avoid lumps formation and to obtain as quickly as possible absorption of the liquid into the cross-linked polyvinylpyrrolidone particles minimizing solvent evaporation. At the end of the wetting and massing the polymer should be completely swollen, appearing as a viscous cream that is quickly transferred into a glass Petri dish. A small sample of swollen product is collected for loss on drying test (LoD), then the Petri dish is transferred into a sealed glass dessiccator containing liquid acetone in equilibrium with its vapor, and it is stored under this organic solvent rich atmosphere for 14-16 hours to allow homogeneous distribution of the solution into the mass of swollen polymer with minimal solvent loss The homogeneous swollen material is then removed from the dessiccator, one sample is collected for solvent quantification and the remaining product is kept (at about room temperature for about 75-90 minutes, unless otherwise specified) under hood to allow evaporation of an aliquot of the solvent without formation of hard crust (pre-drying). The Petri dish is then transferred into a vacuum oven preliminary heated and set-up to maintain internal temperature at 50° C.; samples for solvent quantification are collected. After a while the drying is continued (at about 40° C.) until the composite LoD is comparable or lower than that of the cross-linked polyvinylpyrrolidone measured at process starting (for about 120-180 minutes, unless otherwise specified). The dried composite, eventually manually milled in a ceramic mortar, is transferred into a plastic container closed inside a polyethylene bag and it is stored at room temperature until characterization.

### 3.2 Lab Scale Method

Batch size of the composite is 150 g, unless otherwise specified; process details and samples codes applied to the batches manufactured with this method are listed in Table 4. The required amounts of drug and water and organic solvent soluble polymer are dissolved into organic solvent as previously described. In the meantime the required amount of cross-linked polyvinylpyrrolidone is weighed and transferred into the container of a 1.5 liters low shear twin arms mixer/granulator (Battaggion IP1.5/T); granulator lid is tightly closed then mixing is started and the organic solution previously prepared is added to the cross-linked polyvinylpyrrolidone using peristaltic pump (Flocon 1003) at rate selected to complete liquid distribution in 10-15 minutes. The wet material is massed for 30 minutes at room temperature switching each 10 minutes mixing arms rotation direction, then one sample is collected for loss on drying test. Wet material massing is continued for other ninety minutes at room temperature in presence of acetone vapors to allow homogenous distribution of the solution into the swollen polymer mass. An aliquot of solvent has to be removed from the homogeneous swollen composite (viscous and creamy) to obtain a wet powder easier to quantitatively transfer into vacuum oven for solvent removal completion. Pre-drying in Battaggion is conducted increasing the granulator container temperature by circulation of thermal liquid at about 50° C., reducing the pressure by vacuum pump (Rietschle) connected to solvent recovery system liquid cooled at 5° C., and keeping the product under mixing to speed-up solvent removal and to reduce lump formation. The pre-drying duration is about 40 minutes, unless otherwise specified. Then, the partially dried composite is loaded on one tray that is transferred into vacuum oven (Vuototest, Mazzali) preliminarily heated, set-up to maintain internal temperature of 55° C. and connected with the same pump and solvent recovery system used for the granulator. Drying is continued (for about 120 minutes, unless otherwise specified), until loss on drying value similar or lower than that of CPVP is measured.

### 3.3 Enlarged Lab-Scale Method

Batch size of the composite is 1800 g; details about batches manufactured are reported in Table 4. The process is conducted as described for lab-scale method in section 3.2, with the following changes: A) ten liters low shear twin arms mixer/granulator (Battaggion IP10) and Watson Marlowe peristaltic pump are used; B) pre-drying step into granulator is carried out with heating liquid temperature at 55° C., instead than at 50° C. because of the large volume of the granulator chamber (for about 35 minutes, unless otherwise specified); C) final drying in vacuum oven is conducted at 50° C. (for about 360 min, unless otherwise specified) and the product is distributed onto four trays.

### 4. Fenofibrate Composites Preparation

### 4.1. Fenofibrate High Drug Load Composites (20% and 25%)

4.1.1 REFERENCE 1, REFERENCE 2, SAMPLE 1, SAMPLE 2, SAMPLE 3, SAMPLE 4, SAMPLE 5, SAMPLE 7: these composites have 20% drug load and they are manufactured at 10 g batch size with the manual process described in section 3.1. Process details are shown in Table 2. Fenofibrate/cross-linked polyvinylpyrrolidone weight ratio is 1:4 in all the binary and 1:3 in all the ternary composites. Acetone/cross-linked polyvinylpyrrolidone weight ratio is about 2.3 both in binary and ternary composites. In all the ternary composites fenofibrate/water and organic solvent soluble polymer weight ratio is 1:1.

For preparation of all these composites about 2.0 g of fenofibrate are weighed and dissolved in about 18.5 g (binary composite) or in about 14.0 g (ternary composite) of acetone. In ternary composites about 2.0 g of water and organic solvent soluble polymer are dissolved or dispersed into the fenofibrate/acetone solution.

Solubilization of dimethylaminoethyl methacrylate-butylmethacrylate-methylmethacrylate copolymer (Eudragit® E) (SAMPLE 5) in acetone is longer (about 15 minutes) than that of equivalent amount (2 g) of vinylpyrrolidone-vinyl acetate (Kollidon® VA64) (SAMPLE 1, SAMPLE 6, SAMPLE 7) and polyethyleneglycol-caprolactame-vinylpyrrolidone copolymer (Soluplus®—SAMPLE 3) (2-3 minutes). The amount of cross-linked polyvinylpyrrolidone is about 8.0 g and 6.0 g respectively in binary and ternary composites.

4.1.2 REFERENCE 3, SAMPLE 6: these composites have 25% drug load and they are manufactured at 10 g batch size with the manual process described in section 3.1. Fenofibrate: cross-linked polyvinylpyrrolidone weight ratio is 1:2 in the ternary and 1:3 in the binary composite. Acetone/cross-linked polyvinylpyrrolidone weight ratio is about 2.3 both in binary and ternary composites. In the ternary composite fenofibrate/water and organic solvent soluble polymer ratio is 1:1.

For the preparation of these composites about 2.5 g of fenofibrate are weighed and dissolved in about 17.5 g (binary composite) or in about 11.5 g (ternary composite) of acetone. In ternary composites about 2.0 g of water and organic solvent soluble polymer are dissolved or dispersed into the fenofibrate/acetone solution. The amount of cross-linked polyvinylpyrrolidone is about 7.5 g and 5.0 g respectively in binary and ternary composites. The dried ternary composite is reduced to a fine powder before characterization tests.

### 4.2 Fenofibrate Low Drug Load Composites (10%, 5%)

4.2.1 REFERENCE 4, SAMPLE 8: these composites have 10% drug load and they are manufactured at 15 g batch size with the manual process described in section 3.1. Fenofibrate: cross-linked polyvinylpyrrolidone weight ratio is 1:8 in the ternary and 1:9 in the binary composite. Acetone/cross-linked polyvinylpyrrolidone weight ratio is about 2.3 both in binary and ternary composites. In the ternary composite fenofibrate/water and organic solvent soluble polymer ratio is 1:1.

For the preparation of these composites about 1.5 g of fenofibrate are weighed and dissolved in about 31.0 g (binary composite) or in about 27.5 g (ternary composite) of acetone. In ternary composites about 1.5 g of vinylpyrrolidone-vinyl acetate copolymer (Kollidon® VA64) are into the fenofibrate/acetone solution.

The amount of cross-linked polyvinylpyrrolidone is about 13.5 g and 12.0 g in binary and ternary composites respectively. The dried ternary composite is reduced to a fine powder before characterization tests.

4.2.2 REFERENCE 5, REFERENCE 6, SAMPLE 9, SAMPLE 10: these composites have 5% drug load and they are manufactured at 10 g batch size with the manual process described in section 3.1. Process details are shown in Table 3. Fenofibrate: cross-linked polyvinylpyrrolidone weight ratio is 1:18 in the ternary and 1:19 in the binary composite. Acetone/cross-linked polyvinylpyrrolidone weight ratio is about 2.3 both in binary and ternary composites. In the ternary composite fenofibrate/water and organic solvent soluble polymer ratio is 1:1.

For preparation of these composites about 0.5 g of fenofibrate are weighed and dissolved in about 22.0 g (binary composite) or in about 20.5 g (ternary composite) of acetone. In all ternary composites about 0.5 g of vinylpyrrolidone-vinyl acetate copolymer (Kollidon® VA64) are dissolved into the fenofibrate/acetone solution. The amount of cross-linked polyvinylpyrrolidone is about 9.5 g and 9.0 g respectively in binary and ternary composites. The dried ternary composite is reduced to a fine powder before characterization tests.

### 4.3 Fenofibrate Composites Manufactured on Lab-Scale and Enlarged Lab Scale

4.3.1 REFERENCE 7, SAMPLE 11, SAMPLE 13: these composites have 20% drug load, they are manufactured at 150 g batch size in 1.5 liters low shear mixer-granulator Battaggion IP1.5/T as described in section 3.2 Process details are shown in Table 4, see also Table 9 for SAMPLE 11. Drug/carrier weight ratio is 1:4 in binary and 1:3 in ternary. Acetone/cross-linked polyvinylpyrrolidone weight ratio is about 2.3 both in binary and ternary composites. In the ternary composite fenofibrate/water and organic solvent soluble polymer ratio is 1:1.

For the preparation of these composites about 30 g of fenofibrate are weighed and dissolved in about 276 g (binary composite) or in about 207 g (ternary composite) of acetone. In all ternary composites about 30 g of water and organic solvent soluble polymer are dissolved into the fenofibrate/acetone solution. The water and organic solvent soluble polymer is vinylpyrrolidone-vinyl acetate copolymer in SAMPLE 11 and dimethylaminoethyl methacrylate-butylmethacrylate-methylmethacrylate copolymer in SAMPLE 13. The amount of cross-linked polyvinylpyrrolidone is about 120 g and 90 g respectively in binary and ternary composites.

4.3.2 SAMPLE 12: this composite containing vinylpyrrolidone-vinyl acetate copolymer is manufactured at 1,800 g batch size in low shear mixer-granulator Battaggion IP10 with the enlarged lab-scale process as described in section 3.3. Drug/carrier weight ratio is 1:4 in binary and 1:3 in ternary. Acetone/cross-linked polyvinylpyrrolidone weight ratio is about 2.3 both in binary and ternary composites. In the ternary composite fenofibrate/water and organic solvent soluble polymer ratio is 1:1.

For the preparation of these composites about 360 g of fenofibrate are weighed and dissolved in about 2,485 g of acetone; then about 360 g of vinylpyrrolidone-vinyl acetate copolymer are dissolved into the fenofibrate/acetone solution. The amount of cross-linked polyvinylpyrrolidone is about 1,080 g.

### 5. Nifedipine Composites Preparation

5.1 REFERENCE 8, SAMPLE 14, SAMPLE 15: these composites have 20% drug load and they are manufactured at 15 g batch size with the manual process described in section 3.1. Process details are shown in Table 5. Nifedipine/cross-linked polyvinylpyrrolidone weight ratio is 1:4 and 1:3 respectively in binary and ternary composite. Acetone/cross-linked polyvinylpyrrolidone weight ratio is about 2.3 both in binary and ternary composites. In ternary composites nifedipine/water and organic solvent soluble polymer weight ratio is 1:1.

For preparation of all these composites about 3.0 g of nifedipine are weighed and dissolved in about 27.5 g (binary composite) or in about 20.5 g (ternary composite) of acetone. In ternary composites about 3.0 g of water and organic solvent soluble polymer are dissolved into the nifedipine/acetone solution.

The water and organic solvent soluble polymers used in ternary composites are vinylpyrrolidone-vinyl acetate copolymer and dimethylaminoethyl methacrylate-butylmethacrylate-methylmethacrylate copolymer respectively in SAMPLE 14 and SAMPLE 15. The amount of cross-linked polyvinylpyrrolidone is about 12.0 g and 9.0 g respectively in binary and ternary composites.

### 6. Nimesulide Composites Preparation

6.1 SAMPLE 16, REFERENCE 9: these composites have 16.7% drug load and they are manufactured at 12 g batch size with the manual process described in section 3.1. Process details are shown in Table 5. Nimesulide/cross-linked polyvinylpyrrolidone weight ratio is 1:5 and 1:4 respectively in binary and ternary composite. Acetone/cross-linked polyvinylpyrrolidone weight ratio is about 1.35 both in binary and ternary composites. In ternary composites nimesulide/N-vinylpyrrolidone/vinyl-acetate copolymer weight ratio is 1:1.

For the preparation of all these composites about 2.0 g of nimesulide are weighed and dissolved in about 13.5 g (binary composite) or in about 11.0 g (ternary composite) of acetone. In ternary composite about 2.0 g of vinylpyrrolidone-vinyl acetate copolymer are dissolved into the nifedipine/acetone solution. The amount of cross-linked polyvinylpyrrolidone is about 10.0 g and 8.0 g respectively in binary and ternary composites.

For solubilization kinetic comparison purposes, physical blend was prepared (REFERENCE 10) consisting of the binary composite (REFERENCE 9) and vinylpyrrolidone-vinyl acetate copolymer in amounts resulting in the 1:4:1 ratio of the ternary composite. The blend is prepared by weighing the required amounts REFERENCE 9 and vinylpyrrolidone-vinyl acetate copolymer into a test tube, closed with screw cap and mixing into a Turbula T2C blender for 15 minutes at 25 rpm.

TABLE 2
Details on fenofibrate composites (20% and 25% drug load) prepared by the manual method;

| Drug/carrier/(water | | | | | LoD (%) after | |
|---|---|---|---|---|---|---|
| and organic solvent | Water and | | Theoretical acetone | | swollen | LoD (%) of |
| soluble polymer) (% | organic solvent | | content (% wet | LoD (%) after | composite | dried |
| w/w) | soluble polymer | Sample code | weight) | swelling | homogenization | composite |
| 1:4 | — | REFERENCE 1 | 64.8 | −59.2 | −55.0 | −0.98 |
| (20%) | | | | | | |
| 1:3:1 | Vinylpyrrolidone-vinyl | SAMPLE 1 | 58.0 | −54.8 | ND | −1.10 |
| (20%) | acetate copolymer | | | | | |
| 1:3:1 | Polyvinylpyrrolidone | SAMPLE 2 | 58.0 | −54.4 | −49.4 | −1.80 |
| (20%) | | | | | | |
| 1:3:1 | Polyethyleneglycol- | SAMPLE 3 | 58.0 | −51.9 | −47.2 | −1.20 |
| (20%) | caprolactame- | | | | | |
| | vinylpyrrolidone | | | | | |
| | copolymer | | | | | |
| 1:3:1 | Polyoxyethylene- | SAMPLE 4 | 58.0 | −53.9 | −50.8 | −1.30 |
| (20%) | polyoxypropylene | | | | | |
| | copolymer | | | | | |
| 1:3:1 | Dimethylaminoethyl | SAMPLE 5 | 58.0 | −53.4 | −45.6 | −1.40 |
| (20%) | methacrylate- | | | | | |
| | butylmethacrylate- | | | | | |
| | methylmethacrylate | | | | | |
| | copolymer | | | | | |
| 1:3 | — | REFERENCE 3 | 63.3 | −61.7 | −51.63 | −1.20 |
| (25%) | | | | | | |
| 1:2:1 | Vinylpyrrolidone-vinyl | SAMPLE 6 | 53.5 | 48.0 | −45.0 | −0.71 |
| (25%) | acetate copolymer | | | | | |
| 1:4 | — | REFERENCE 2 | 64.8 | −62.1 | −52.7 | −2.00 |
| (20%) | | | | | | |
| 1:3:1 | Vinylpyrrolidone-vinyl | SAMPLE 7 | 58.0 | −54.1 | ND | −1.40 |
| (20%) | acetate copolymer | | | | | |

ND: not determined

TABLE 3
Details on the manual method production of fenofibrate composites (10% and 5% drug load)

| Drug/carrier/ | | | | | | |
|---|---|---|---|---|---|---|
| (water and | | | | | | |
| organic | | | Theroretical | | LoD (%) | |
| solvent | | | acetone | | after | |
| soluble | Water and | | content | LoD (%) | swollen | LoD (%) |
| polymer) | organic solvent | | (% wet | after | composite | of dried |
| (% w/w) | soluble polymer | Sample code | weight) | swelling | homogenization | composite |
| 1:9 | — | REFERENCE 4 | 67.4 | −65.1 | −57.8 | −0.8 |
| (10.0%) | | (15 g) | | | | |
| 1:19 | — | REFERENCE 5 | 68.6 | −65.2 | −60.2 | −1.1 |
| (5.0%) | | (10 g) | | | | |
| | | REFERENCE 6 | | −64.7 | −61.1 | −1.3 |
| | | (10 g) | | | | |
| 1:8:1 | Vinylpyrrolidone-vinyl | SAMPLE 8 | 64.8 | −63.0 | −59.1 | −1.9 |
| (10.0%) | acetate copolymer | (15 g) | | | | |
| 1:18:1 | Vinylpyrrolidone-vinyl | SAMPLE 9 | 67.4 | −63.3 | −57.8 | −1.2 |
| (5.0%) | acetate copolymer | (10 g) | | | | |
| | | SAMPLE 10 | | −63.9 | −59.9 | −1.2 |
| | | (10 g) | | | | |

TABLE 4
Details on fenofibrate composites prepared by the lab-scale and enlarged lab-scale methods

| Drug/carrier/ | | | | | | |
|---|---|---|---|---|---|---|
| (water and | | | | | | |
| organic | Water and | | Theoretical | | LoD (%) | |
| solvent | organic | | acetone | | after | |
| soluble | solvent | | content | LoD (%) | swollen | LoD (%) |
| polymer) | soluble | | (% wet | after | composite | of dried |
| (% w/w) | polymer | Sample code | weight) | swelling | homogenization | composite |
| 1:4 | — | REFERENCE 7 | 64.8 | −62.6 | −60.8 | −1.0 |
| (20%) | | (150 g) | | | | |
| 1:3:1 | Vinylpyrrolidone-vinyl | SAMPLE 11 | 58.0 | −56.9 | −55.4 | −0.6 |
| (20%) | acetate copolymer | (150 g) | | | | |
| 1:3:1 | Vinylpyrrolidone-vinyl | SAMPLE 12 | 58.0 | −58.7 | −57.5 | −2.2 |
| (20%) | acetate copolymer | (1800 g) | | | | |
| 1:3:1 | Dimethylaminoethyl | SAMPLE 13 | 58.0 | ND | 53.4 | −1.0 |
| (20%) | methacrylate- | (150 g) | | | | |
| | butylmethacrylate- | | | | | |
| | methylmethacrylate | | | | | |
| | copolymer | | | | | |

TABLE 5
Details on nifedipine and nimesulide composites prepared by the manual method

| Drug/carrier/ | | | | | | | |
|---|---|---|---|---|---|---|---|
| (water and | | | | | | | |
| organic | | | | Theoretical | | LoD (%) | |
| solvent | | | | acetone | | after | |
| soluble | | Water and | | content | LoD (%) | swollen | LoD (%) |
| polymer) | | organic solvent | | (% wet | after | composite | of dried |
| (% w/w) | Drug | soluble polymer | Sample code | weight) | swelling | homogenization | composite |
| 1:4 | Nifedipine | — | REFERENCE 8 | 64.8 | −61.89 | −60.49 | −1.85 |
| (20%) | | | | | | | |
| 1:3:1 | Nifedipine | Vinylpyrrolidone-vinyl | SAMPLE 14 | 58.0 | −58.33 | −52.41 | −2.25 |
| (20%) | | acetate copolymer | | | | | |
| 1:3:1 | Nifedipine | Dimethylaminoethyl | SAMPLE 15 | 58.0 | −53.98 | −51.71 | −1.60 |
| (20%) | | methacrylate- | | | | | |
| | | butylmethacrylate- | | | | | |
| | | methylmethacrylate | | | | | |
| | | copolymer | | | | | |
| 1:5 | Nimesulide | — | REFERENCE 9 | 52.9 | −50.21 | −40.21 | −0.27 |
| (16.7%) | | | | | | | |
| 1:4:1 | Nimesulide | Vinylpyrrolidone-vinyl | SAMPLE 16 | 47.7 | −40.97 | −39.40 | −0.54 |
| (16.7%) | | acetate copolymer | | | | | |

TABLE 9
Production and drying details of composites prepared with the lab-scale equipment
(20% drug load); composite components: fenofibrate cross-linked polyvinylpyrrolidone
(Kollidon ® CLM)/vinylpyrrolidone-vinyl acetate copolymer (Kollidon ® VA64)

| Drug/ | | | | | | |
|---|---|---|---|---|---|---|
| carrier/ | | | Polymeric carrier swelling and homogenization step | | | |
| (water and | | | | | Duration | LoD (%) |
| organic | Water and | | Theoretical | | of swollen | after |
| solvent | organic | | acetone | LoD (%) | composite | swollen |
| soluble | solvent | | content | after | homogen- | composite |
| polymer) | soluble | | (% wet | solution | ization | homogen- |
| (% w/w) | polymer | Sample code | weight) | distribution | (min) | ization |
| 1:4 | — | REFERENCE 7 | 64.8 | −62.6 | 90 | −60.8 |
| (20%) | | | | | | |
| 1:3:1 | Vinylpyrrol | SAMPLE 11 | 58.0 | −56.9 | 90 | −55.4 |
| (20%) | idone- | | | | | |
| | vinyl | | | | | |
| | acetate | | | | | |
| | copolymer | | | | | |

| Drug/ | | | | | | | |
|---|---|---|---|---|---|---|---|
| carrier/ | | | | | | | |
| (water and | | | | | | | |
| organic | | | | Drying step | | | |
| solvent | Pre-drying step | | | | | | LoD (%) |
| soluble | | Heater | | | Oven | | of |
| polymer) | Time | temp. | Pressure | Duration | temp | Pressure | dried |
| (% w/w) | (min) | (° C.) | (bar) | (min) | (° C.) | (bar) | composite |
| 1:4 | 40 | 50 | 0.25-0.35 | 180 | 52-54 | 0.30-0.35 | −1.0 |
| (20%) | | | | | | | |
| 1:3:1 | 40 | 50 | 0.25-0.40 | 120 | 52-54 | 0.25-0.30 | −0.6 |
| (20%) | | | | | | | |

TABLE 10
Production and drying details of composites obtained with enlarged lab scale equipment (20% drug load);
composite cross-linked polyvinylpyrrolidone (Kollidon ® CLM)/vinylpyrrolidone-vinyl acetate
copolymer (Kollidon ® VA64) 1:3:1;

| | Polymer swelling and homogenization step | | | | | | | |
|---|---|---|---|---|---|---|---|---|
| | Theoretical | | Duration | | | | | |
| | acetone | | of swollen | LoD (%) | Pre-drying step | | | |
| components | content | LoD (%) | composite | after swollen | | Heater | | |
| fenofibrate/ | (% wet | after | homogenization | composite | Duration | temp. | Pressure | |
| Series | weight) | swelling | (min) | homogenization | (min) | (° C.) | (bar) | LoD (%) |
| A | 58.0 | −59.9 | 90 | −59.5 | 90 | 55 | 0.25-0.35 | −6.2 |
| B | 58.0 | −58.7 | 90 | −57.5 | 35 | 55 | 0.30-0.35 | −40.4 |

| | Drying step | | | | | | |
|---|---|---|---|---|---|---|---|
| | | | | | | Process | |
| | | | | | | air | |
| | | | | | Pressure | humidity | |
| | | | | | (oven and | (fluid | |
| components | Sub-series | | | | low | bed) (g | LoD (%) |
| fenofibrate/ | (heating | Sample | Duration | Temp | shear) | H2O/Kg | of dried |
| Series | method) | code | (min) | (° C.) | (bar) | air) | composite |
| A | A1* | SAMPLE | 420 | N.D. | 0.25-0.20 | Not | −1.6 |
| | | 17a | | | | applic | |
| | A2° | SAMPLE | 360 | 40-56 | 0.30-0.25 | Not | −1.9 |
| | | 17b | | | | applic | |
| | A3\hat{ } | SAMPLE | 180 | 43-57 | Not | 0.3-0.4 | −0.5 |
| | | 17c | | | applic. | | |
| B | B1* | SAMPLE | 420 | 32-46 | 0.25-0.20 | Not | −1.9 |
| | | 12a | | | | applic | |
| | B2° | SAMPLE | 360 | 40-52 | 0.30-0.25 | Not | −2.2 |
| | | 12b | | | | applic | |
| | B3\hat{ } | SAMPLE | 180 | 44-55 | Not | 0.3-0.4 | −0.5 |
| | | 12c | | | applic. | | |

*low shear granulator;
°vacuum oven,
\hat{ }fluid bed;
N.D.: not determined

### 7. Fenofibrate Composites Characterization

### 7.2 Solid State Properties of Composites

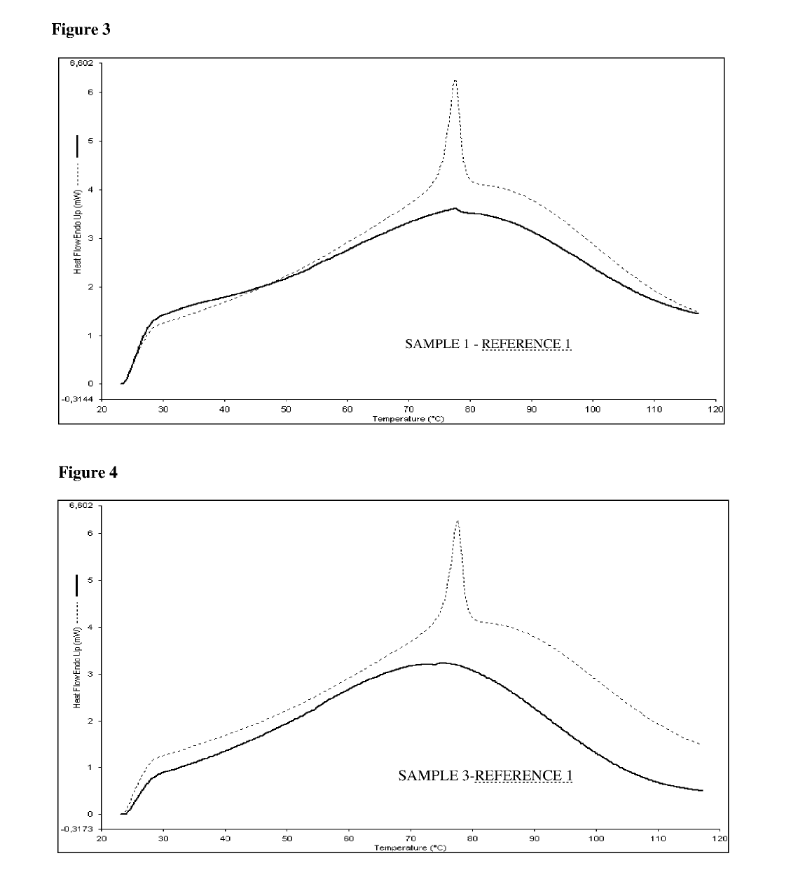

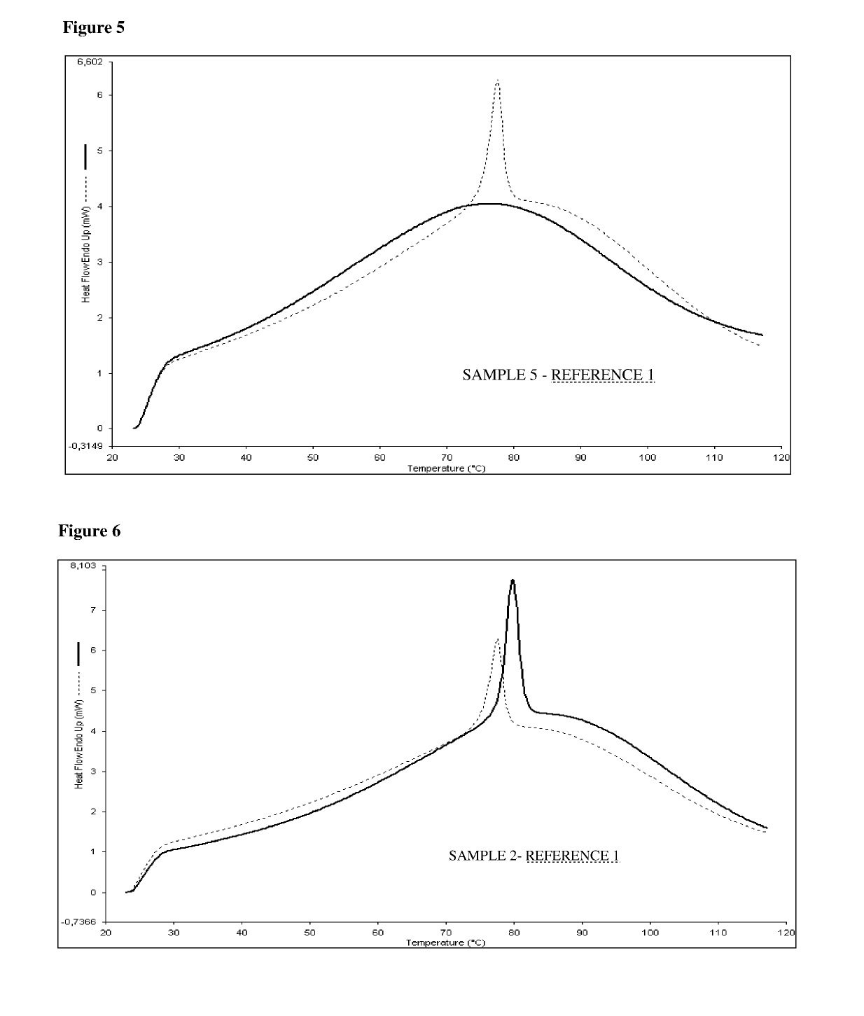

In the DSC traces of 20% drug load ternary composites containing vinylpyrrolidone-vinyl acetate copolymer (SAMPLE 1, FIG. 3), polyethyleneglycol-caprolactame-vinylpyrrolidone copolymer copolymer (SAMPLE 3, FIG. 4), dimethylaminoethyl methacrylate-butylmethacrylate-methylmethacrylate copolymer (SAMPLE 5, FIG. 5) no evidence of fenofibrate melting is visible. Therefore, considering that drug/excipient interactions induced by DSC scan conditions have been previously excluded (in preliminary analysis of drug/excipients blends, not herein reported), it is assumed that in these composites all the fenofibrate is in amorphous form.

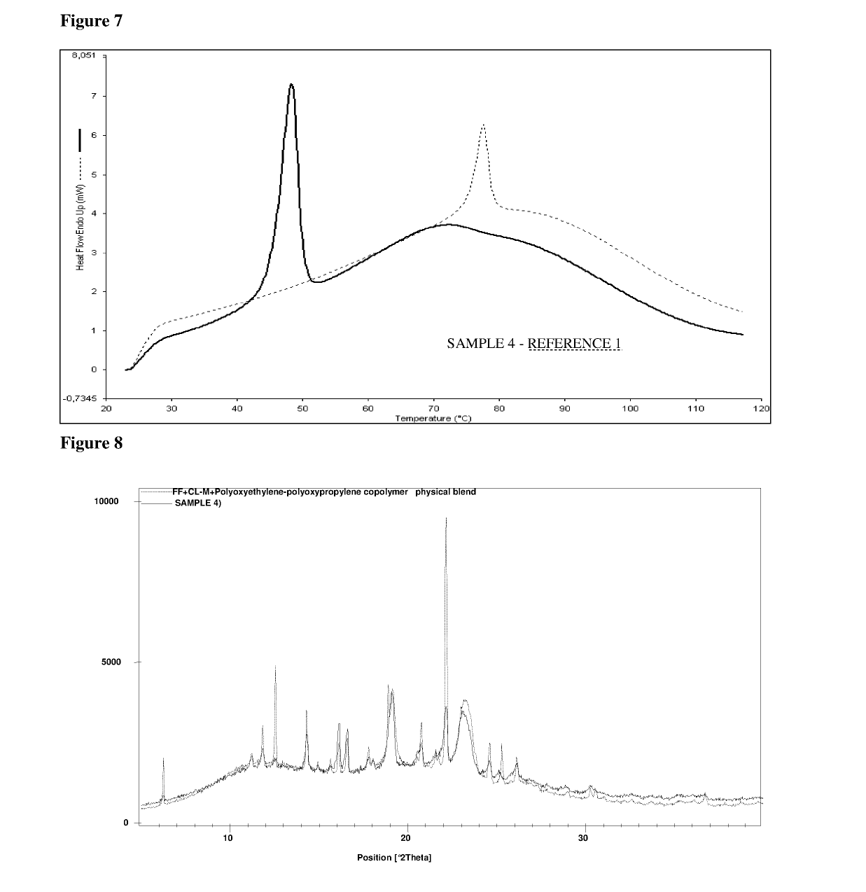

Endothermic event at temperature close to that of fenofibrate melting point is evident in the DSC traces of 20% binary composite (REFERENCE 1) and in that of the 20% ternary composite containing polyvinylpyrrolidone (SAMPLE 2, FIG. 6). Fenofibrate specific melting enthalpy values associated to these thermal events are lower than that measured for the fusion of pure fenofibrate. Being no interaction between fenofibrate and polyvinylpyrrolidone or between fenofibrate and cross-linked polyvinylpyrrolidone induced by DSC scan conditions, it is assumed that aliquots of amorphous and crystalline fenofibrate are mixed into each of these two composites. Into the DSC trace of 20% composite containing polyoxyethylene-polyoxypropylene copolymer (SAMPLE 4, FIG. 7) no thermal event are visible apart water evaporation from the carrier polymer. Considering that possible interaction between drug and polyoxyethylene polyoxypropylene copolymer have been pointed out with DSC scan of physical blend, this composite is analyzed also by XRPD, confirming the presence of crystalline fenofibrate (FIG. 8) which amount cannot anyway be quantified.

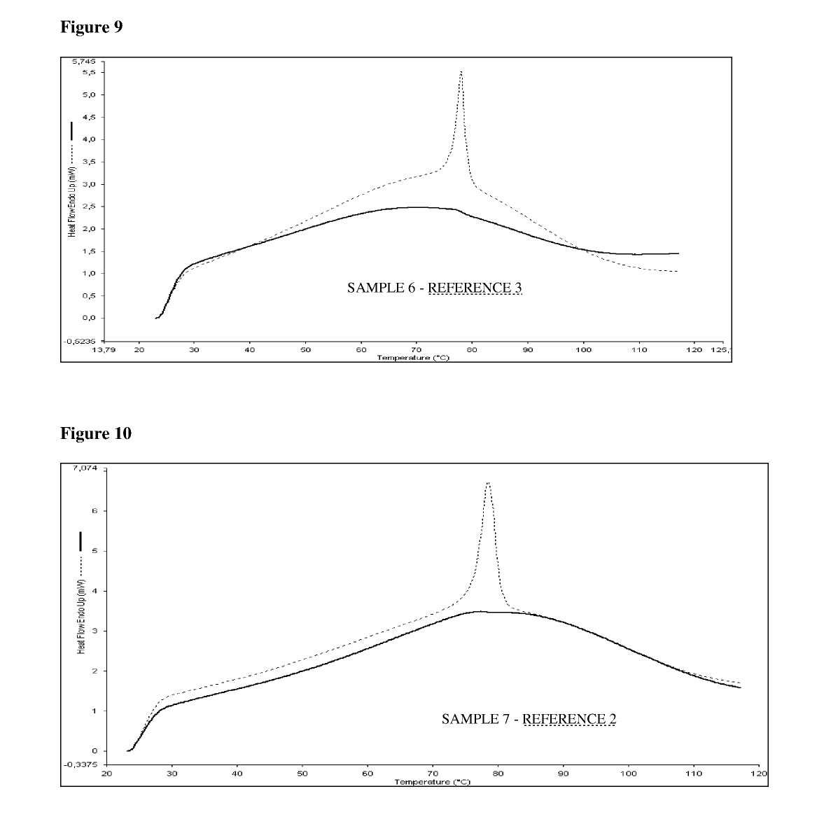

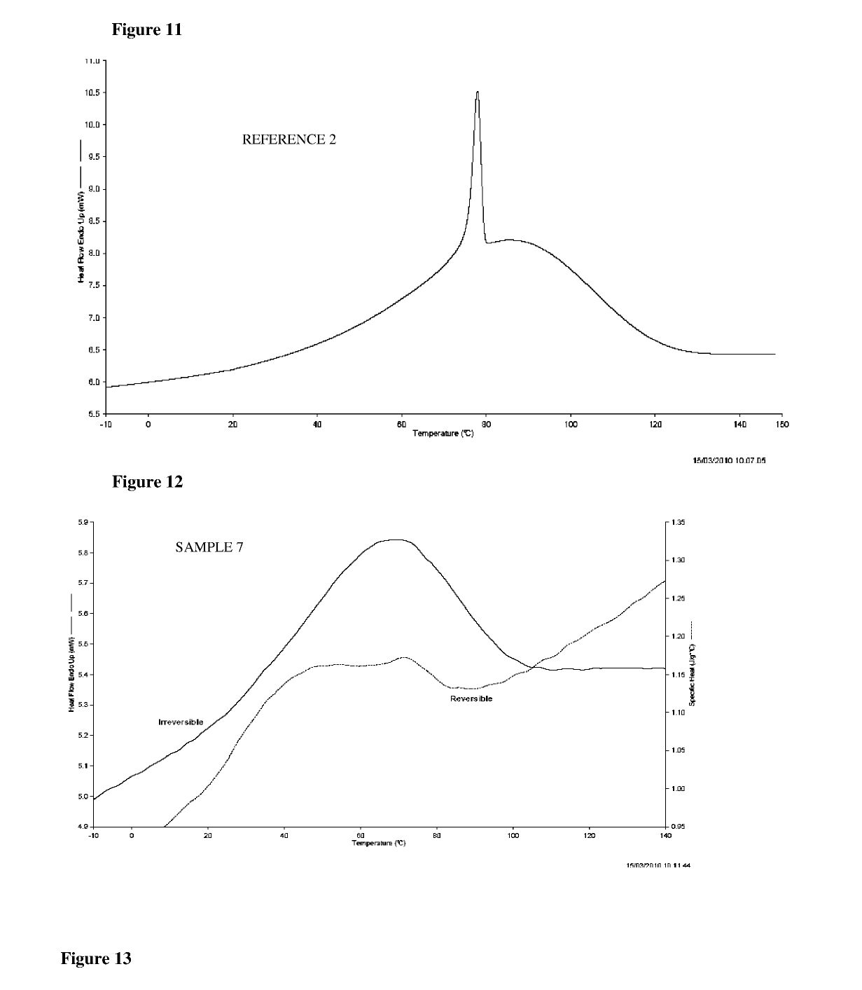

In FIG. 10 the DSC traces of 20% binary (REFERENCE 2) and 20% ternary (SAMPLE 7) composites are qualitatively compared: the ternary composite trace does not contain thermal event corresponding to drug melting in agreement with quantitative analysis finding. These two samples have also been analyzed by quantitative DSC. Composite with 25% drug load show similar solid state properties of corresponding ones at 20% drug load; their DSC traces, compared in FIG. 9 shows that crystalline drug is present in binary composite (REFERENCE 3) and that the ternary composite (SAMPLE 6) contains only amorphous drug.

Quantification of the amount of crystalline material into 20% binary composite and into 20% ternary composite containing vinylpyrrolidone-vinyl acetate copolymer has been conducted on several samples.

For REFERENCE 2 and SAMPLE 7 quantitative DSC analysis results are listed in Table 6 and the corresponding DSC traces acquired with QDSC method are presented in FIGS. 11 (REFERENCE 2) and 13 (SAMPLE 7).

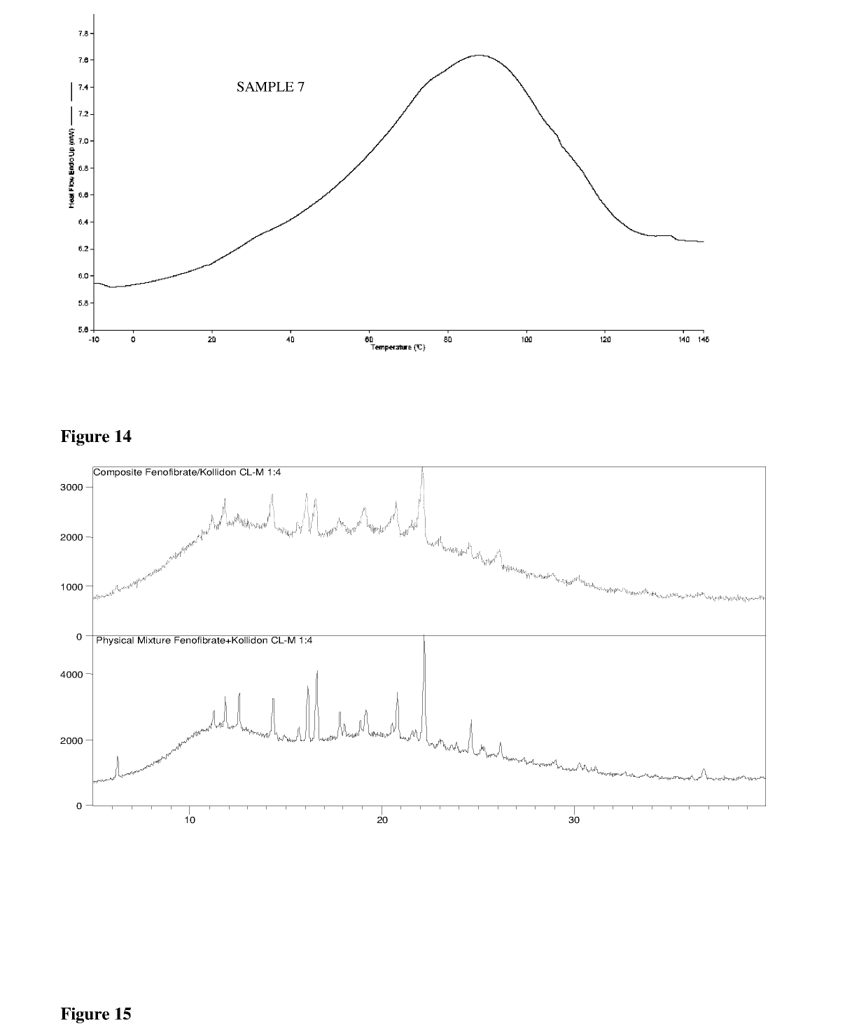

Powder X-Ray Diffraction of REFERENCE 2 binary composite confirms that crystalline fenofibrate is in the same polymorphic form as the starting material (FIG. 14). The endothermic event found into DSC scan (according to XRPD result) of the reference binary composites can be assigned to the fusion of fenofibrate nano-crystals which size distribution (FIG. 15) indicates average size of about 110 nm (estimated from the DSC scan with a dedicated elaboration method). The absence of crystalline fenofibrate into the ternary composite is confirmed by step-scan DSC run of SAMPLE 7. Solid product melting or liquid evaporation are irreversible events; in the step-scan DSC trace presented in FIG. 12 only the broad endotherm caused by water evaporation is visible in the irreversible curve; in the reversible curve one small thermal event, very likely a glass transition, is visible at about 75° C. According to this “events separation”, it is possible to conclude that the very small hump present at about 75° C. into the standard DSC scan of fenofibrate ternary composites containing vinylpyrrolidone-vinyl acetate copolymer (FIG. 3, FIG. 10, FIG. 13) is not caused by the melting of residual aliquot of crystalline drug.

TABLE 6
Summary of the solid state quantitative analysis of composites SAMPLE 7 and
REFERENCE 2.

| | | | | | Crystalline fenofibrate | |
|---|---|---|---|---|---|---|
| | | | | | content (QDSC) | |
| | | Fenofibrate content | | | Relative | |
| | | (assay) | | | residual | |
| | | Theoretical | Measured | % of | crystallinity | Stdev |
| Composite | Sample code | (mg FF/g) | (mg FF/g) | theoretical | (%) | of RRC |
| FF+ cross-linked | REFERENCE 2 | 200.0 | 190.9 | 95.5 | 46 | 2.9 |
| polyvinylpyrrolidone | | | | | | |
| 1:4 | | | | | | |
| FF+ cross-linked | SAMPLE 7 | 200.0 | 193.6 | 96.8 | 0 | 0.0 |
| polyvinylpyrrolidone + | | | | | | |
| vinylpyrrolidone-vinyl | | | | | | |
| acetate copolymer | | | | | | |
| 1:3:1 | | | | | | |

The results of QDSC analysis of the two samples of composite manufactured at 150 g size lab-scale process are listed in Table 7: the ternary composite containing vinylpyrrolidone-vinyl acetate copolymer (SAMPLE 11) shows presence of very little amount of crystalline fenofibrate. On the other hand binary composite (REFERENCE 7) contains smaller amount of crystalline drug than the sample manufactured with manual process. Table 7 contains also results of QDSC test on one sample of ternary composite manufactured at 1.8 kg scale (SAMPLE 12).

TABLE 7
Quantitative solid state analysis of 20% drug loaded ternary
composite containing vinylpyrrolidone- vinyl acetate copolymer,
prepared with lab scale and enlarged lab scale equipments

| | | Crystalline fenofibrate | |
|---|---|---|---|
| | | Content (QDSC) | |
| | | Relative residual | Stdev of |
| Composite | Sample code | crystallinity (%) | RRC |
| FF+ cross-linked | REFERENCE 7 | 26.0 | 0.74 |
| polyvinylpyrrolidone | | | |
| 1:4 | | | |
| FF+ cross-linked | SAMPLE 11 | 0.8 | 0.03 |
| polyvinylpyrrolidone + | | | |
| vinylpyrrolidone- vinyl | | | |
| acetate copolymer | | | |
| 1:3:1 | | | |
| FF+ cross-linked | SAMPLE 12 | 0.0 | 0.00 |
| polyvinylpyrrolidone + | | | |
| vinylpyrrolidone- vinyl | | | |
| acetate copolymer | | | |
| 1:3:1 | | | |

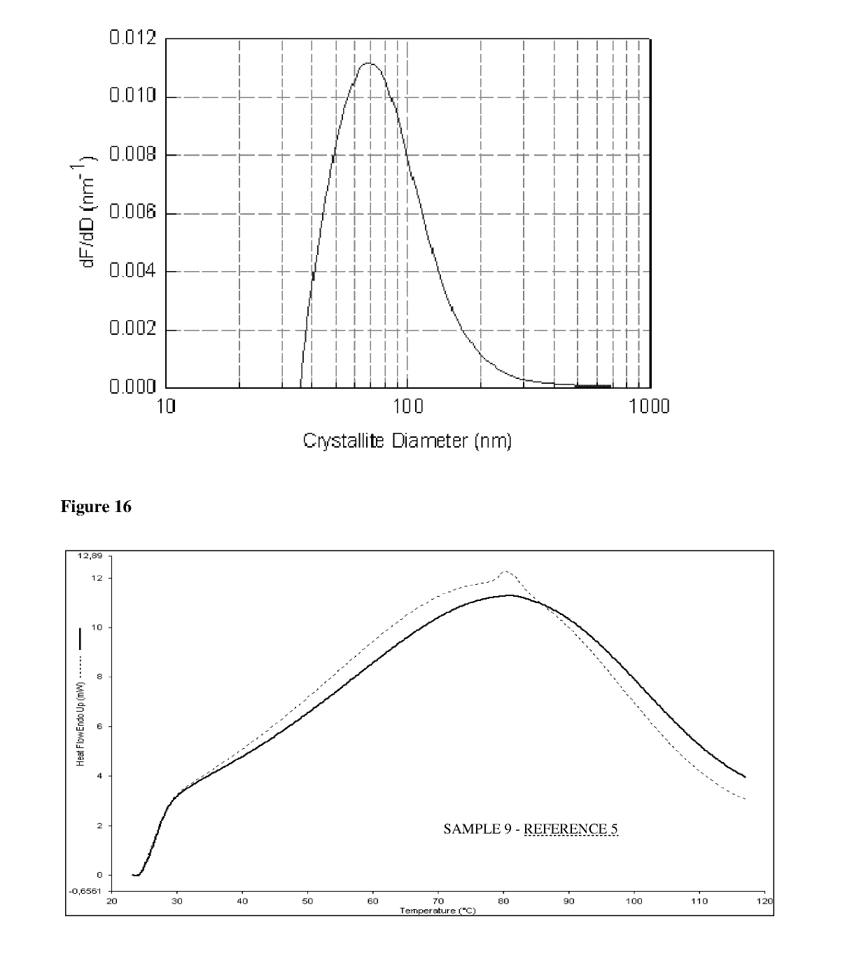

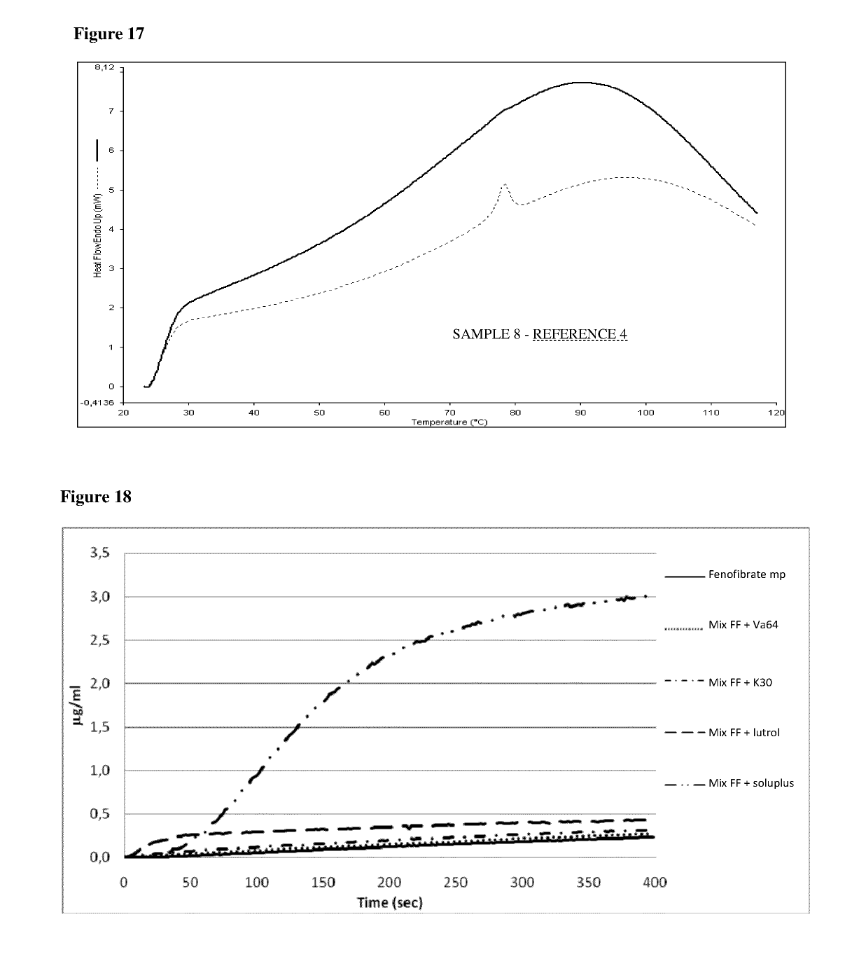

Ternary composites with vinylpyrrolidone-vinyl acetate copolymer prepared with the manual process with drug loads of 10% (1:8:1, SAMPLE 8) and 5% (1:18:1, SAMPLE 9) contain only amorphous fenofibrate, according to their DSC scan (FIGS. 16 and 17) in which no thermal event, apart the water evaporation is detected. This qualitative evaluation is confirmed by QDSC analysis conducted on SAMPLE 10 resulting in 0% residual crystallinity. The DSC scans of the reference binary composites with corresponding drug loads (10%: REFERENCE 4 and 5%: REFERENCE 5) show the fenofibrate melting endotherm, suggesting that even drug load reduction up to 5% is not sufficient to obtain complete transition of the active ingredient to amorphous state (FIG. 16, 17, 18). The amount of crystalline fenofibrate into the low drug loaded binary composite tested (1:19) resulted about 33% of the fenofibrate content, according to QDSC scan conducted on REFERENCE 6. Binary composites containing all the fenofibrate in amorphous form cannot be obtained, whereas, the ternary composites contained only amorphous fenofibrate.

7.3. Solubilization Properties of Composites.

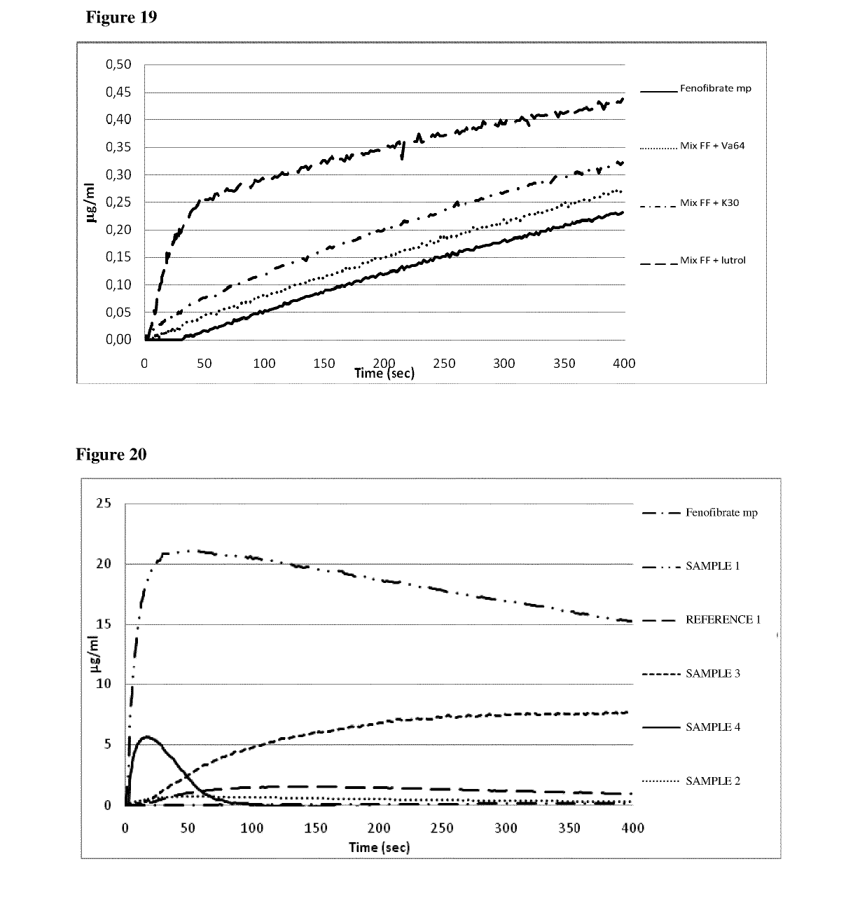

Solubilization kinetic profiles of crystalline fenofibrate raw material as is and blended with one of the water and organic solvent soluble polymers (1:1 weight ratio) are presented in FIGS. 18 and 19. The solubilization profile of fenofibrate is very close to that of its physical blend with vinylpyrrolidone-vinyl acetate copolymer and polyvinylpyrrolidone. Whereas, in presence of two surfactants polymers (polyethyleneglycol-caprolactame-vinylpyrrolidone copolymer and polyoxyethylene-polyoxypropylene copolymer) the solubilization of fenofibrate is promoted, being the SK profiles shifted upward and with different shapes with respect to that of the active ingredient alone. Polyethyleneglycol-caprolactame-vinylpyrrolidone copolymer is more effective than polyoxyethylene-polyoxypropylene copolymer in improving fenofibrate solubility under the test conditions.

In FIG. 20 the SK profiles of 20% ternary composites prepared with the four different water and organic solvent soluble polymers (vinylpyrrolidone-vinyl acetate copolymer: SAMPLE 1, polyvinylpyrrolidone: SAMPLE 2, polyethyleneglycol-caprolactame-vinylpyrrolidone copolymer copolymer: SAMPLE 3 and polyoxyethylene-polyoxypropylene copolymer: SAMPLE 4) are compared with that of the binary composite with corresponding drug load (REFERENCE 1) and with that of fenofibrate raw material.

Fenofibrate solubility peak about 40 times higher than the equilibrium solubility measured for the crystalline drug is obtained in the vinylpyrrolidone-vinyl acetate copolymer ternary composite (SAMPLE 1); in the binary composite of equivalent drug load (REFERENCE 1) the solubility peak is only about 4 times the value of fenofibrate equilibrium solubility (1.6 mcg/ml vs 0.42 mcg/ml). For both these composites the solubility peak is followed by drug concentration decrease which speed is higher in case of the binary composite.

In the SK profile of polyethyleneglycol-caprolactame-vinylpyrrolidone copolymer based ternary composite (SAMPLE 3) a dissolved drug concentration plateau of about 18 times the fenofibrate solubility is reached in about 350 seconds. The shapes the SK profile is different from that of N-vinylpyrrolidone/vinyl-acetate copolymer containing composite. Both vinylpyrrolidone-vinyl acetate copolymer and polyethyleneglycol-caprolactame-vinylpyrrolidone copolymer copolymer based ternary composites contain all the drug in amorphous form (FIG. 3 and FIG. 4).

Into the SK traces of the ternary composite containing polyoxyethylene-polyoxypropylene copolymer (SAMPLE 4) the solubility peak is lower and the drug precipitation rate is faster than in that of vinylpyrrolidone-vinyl acetate copolymer. Solid state analysis indicates that this ternary composite contains both nano-crystalline and amorphous drug even if solid phases quantification is not conducted. From the above, it is clear that the solubility of the not chemically cross-linked polymer in the process solvent is a factor that is important in order to achieve good solubility performance of the composite; same is observed for the solid state property (see section 7.2).

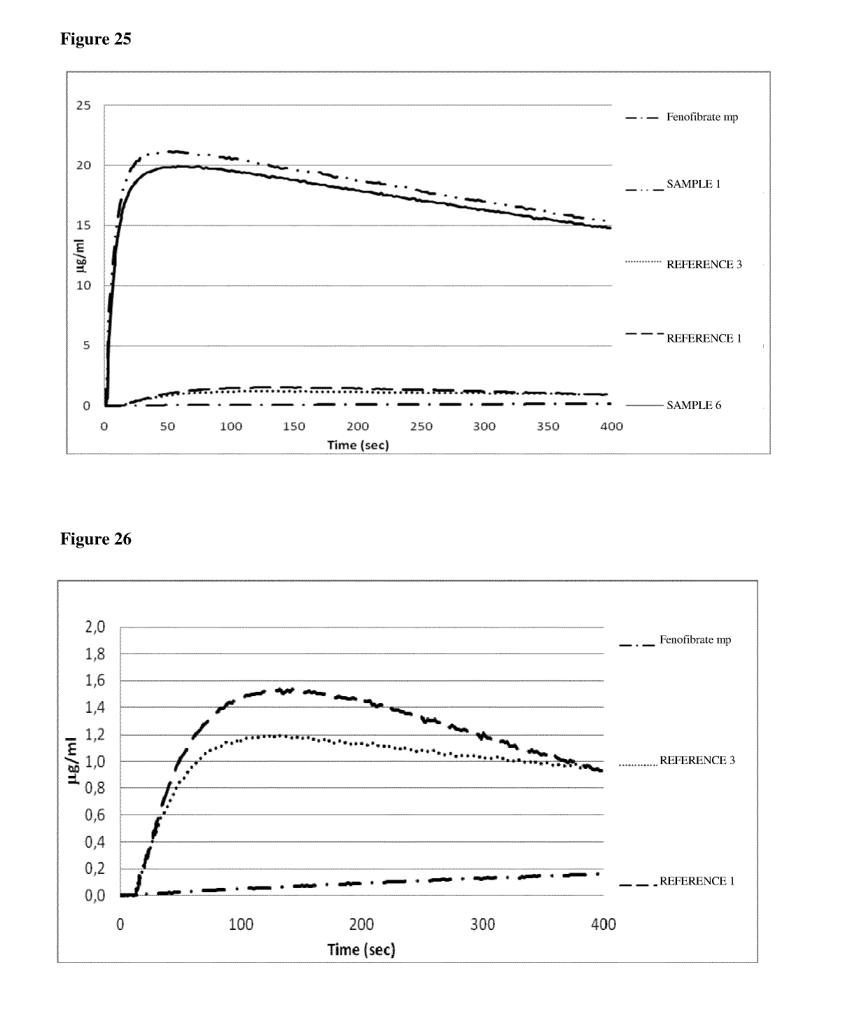

In the case of 25% drug load composites the SK test solubility peak of ternary composite containing vinylpyrrolidone-vinyl acetate copolymer (SAMPLE 6) is higher than that of the corresponding binary (REFERENCE 3), as shown in FIG. 25.

Drug load increase from 20% to 25% results in a slight reduction of the solubility peak values. SK profiles in FIG. 26 show that the difference of the solubility peak value from 20% to 25% drug load composites is higher for binary than for ternary (about 25% versus about 5% of the value).

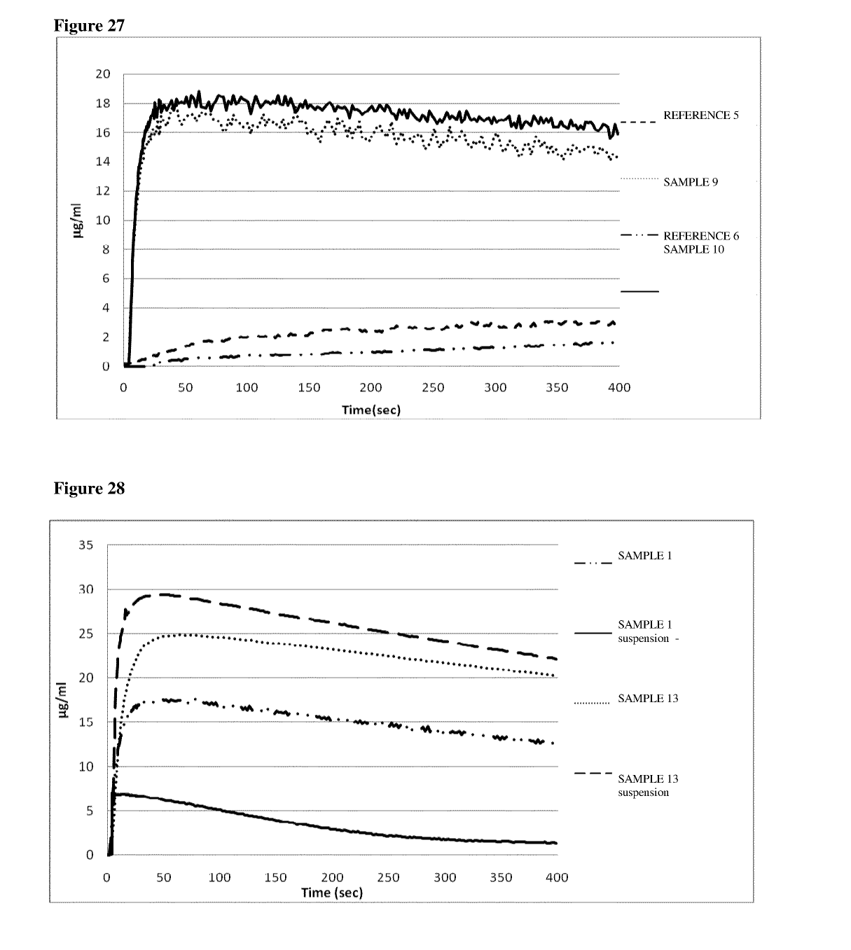

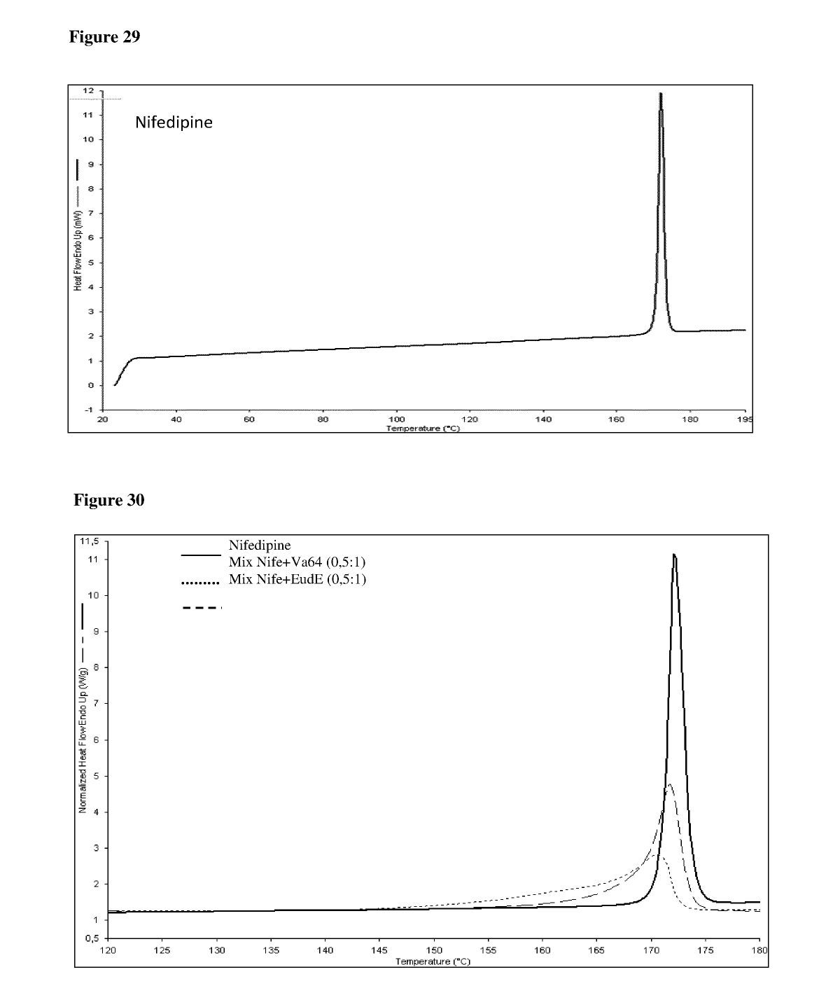

Also 10% and 5% drug load ternary composites with vinylpyrrolidone-vinyl acetate copolymer have solubility enhancement performance superior than corresponding binary as shown in FIG. 24 and FIG. 27 respectively.

The SK tests of 5% loaded composites are measured reducing the oversaturation level from 75 to 40 times the fenofibrate solubility to avoid interference of cross-linked polyvinylpyrrolidone on the UV absorbance of fenofibrate. The comparison can be done only for SK profiles measured applying same oversaturation factor, being the drug “peak solubility” directly proportional to this parameter.

The SK profiles of four samples of 5% drug load composites, two binary (REFERENCE 5 and REFERENCE 6) and two ternary (SAMPLE 9 and SAMPLE 10) manufactured with manual process and batch size of 10 g are compared in FIG. 27. It is clear that, ternary composites have superior solubility enhancement performance than the known binary ones; inter-batch variability observed between two ternary is experimentally acceptable.

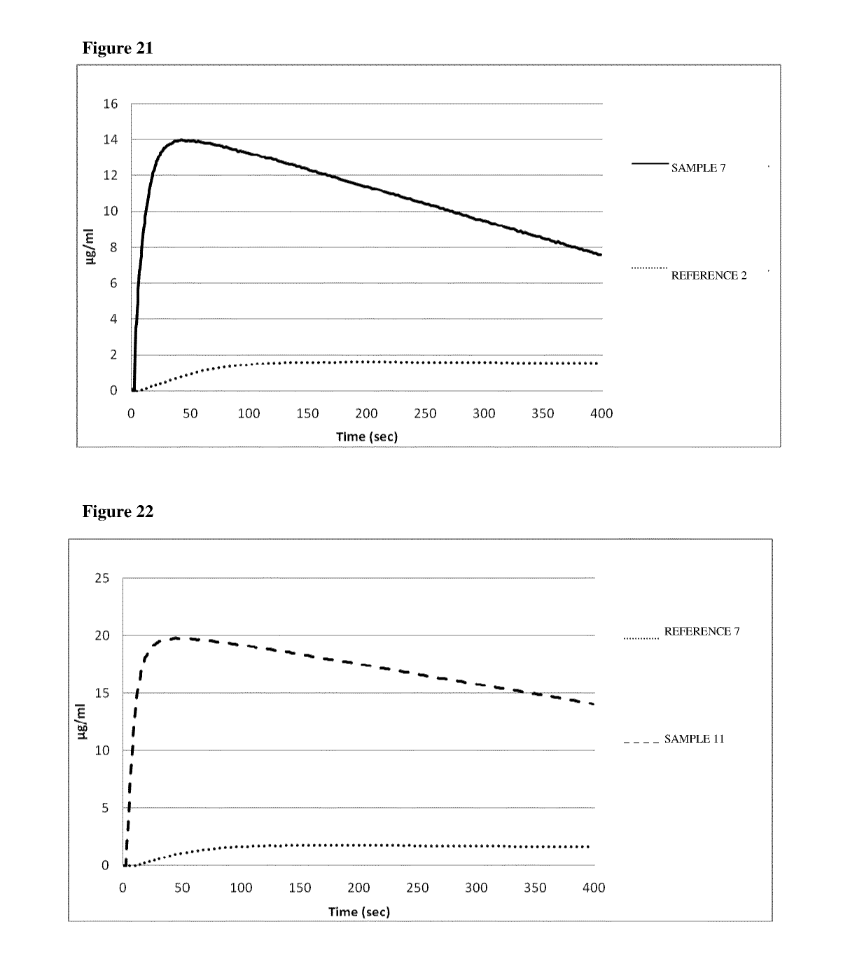

Binary and ternary composites with 20% drug load prepared with lab-scale method at 150 g size (REFERENCE 7 and SAMPLE 11 respectively) have same ratio between SK profiles observed for equivalent composites manufactured with manual process at 10 g size (i.e. REFERENCE 2 and SAMPLE 7). A comparison of SK profiles is shown in FIG. 22 (150 g batch size) and FIG. 21 (10 g batch size).

The SK profile of 20% drug load ternary composites containing dimethylaminoethyl methacrylate-butylmethacrylate-methylmethacrylate copolymer is significantly higher than that of the corresponding binary composite with equivalent drug load as results from the comparison of SAMPLE 13 (FIG. 28) and REFERENCE 2 (FIG. 21) profiles. The SK profile shape is comparable to that of the ternary composite containing vinylpyrrolidone-vinyl acetate copolymer, even if the solubility peak is significantly higher in the case of dimethylaminoethyl methacrylate-butylmethacrylate-methylmethacrylate copolymer (FIG. 28).

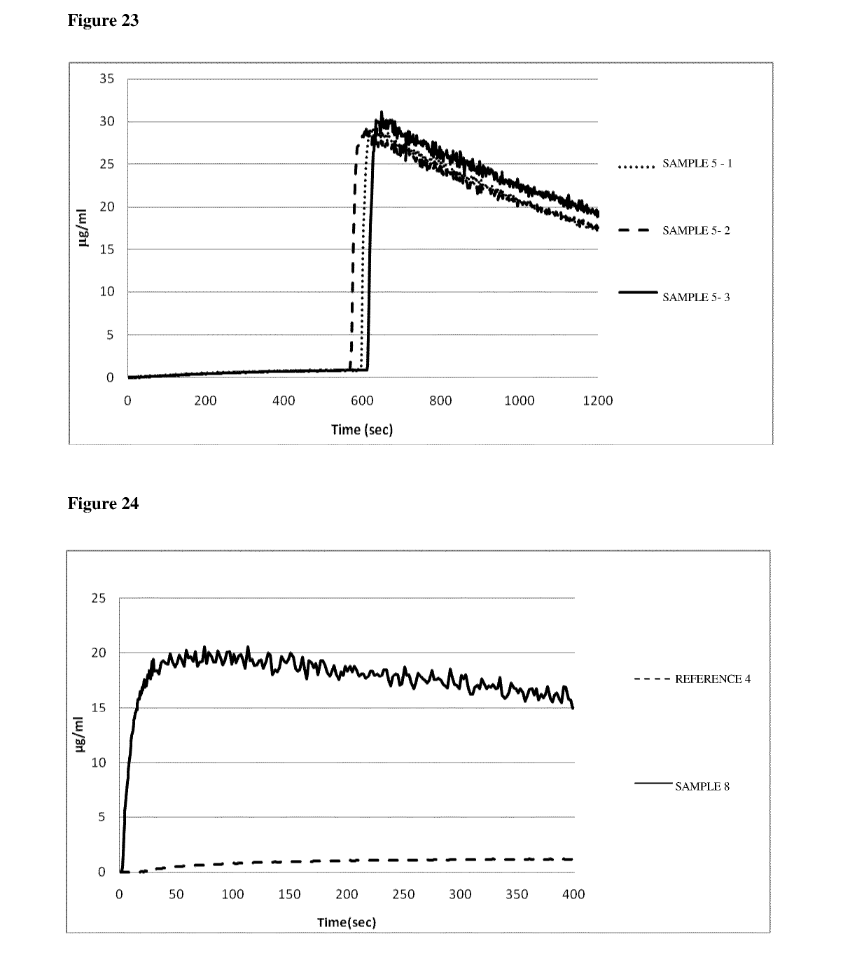

Moreover, the SK test of ternary composite comprising dimethylaminoethyl methacrylate-butylmethacrylate-methylmethacrylate copolymer conducted at pH 1.2 is not impaired by the presence of this polymer that is readily soluble at pH below 5. The “two steps solubilization kinetic test” is applied to verify a possible effect of dimethylaminoethyl methacrylate-butylmethacrylate-methylmethacrylate copolymer on SK profile when a medium at pH above dimethylaminoethyl methacrylate-butylmethacrylate-methylmethacrylate copolymer solubility trigger value is used. FIG. 23 shows that no significant amount of fenofibrate is found in solution during the first ten minutes (phosphate buffer at pH 6.8), followed by a quick release when the pH becomes acidic, and then the obtainment of concentration value equivalent to the solubility peak value measured for similar composite in the standard SK test at pH 1.2 (compare SAMPLE 5 in FIG. 23 and SAMPLE 13 in FIG. 28). The SK profiles obtained with three replications of the “two steps” experiments are well in agreement each other.

In the present invention it is shown that the SK of composites suspended in water for a period of time before the test, is not significantly different than that measured on dry powder when dimethylaminoethyl methacrylate-butylmethacrylate-methylmethacrylate copolymer is used as water and organic solvent soluble polymer. The solubility peak of composite when measured after ten minute of suspension in water is not decreased as it happens in ternary composites containing vinylpyrrolidone-vinyl acetate copolymer. In SAMPLE 13 (containing dimethylaminoethyl methacrylate-butylmethacrylate-methylmethacrylate copolymer) the solubility maximum value is even higher in the SK profile measured after suspension of the samples in water than in that of the powder as is (FIG. 28); in presence of dimethylaminoethyl methacrylate-butylmethacrylate-methylmethacrylate copolymer the suspension foster the dispersion of composite powder lumps and particles aggregate before the dispersion into medium of the SK test. These types of composites may be particularly interesting for preparation of pharmaceutical dosage forms such as sprinkle, dry syrup, extemporaneous suspension, sachets.

### 8. Nifedipine Composites Characterization

### 8.1 Solid State Properties of Active Ingredient, Excipients and Physical Blend.

DSC analysis of physical blends between nifedipine and two water and organic solvent soluble polymers, vinylpyrrolidone-vinyl acetate copolymer and dimethylaminoethyl methacrylate-butylmethacrylate-methylmethacrylate copolymer, have pointed out possibility of interaction induced by DSC scan conditions. A significant shape modification, height reduction and temperature shift of the peak corresponding to nifedipine melting is evident in physical blends of the drug with vinylpyrrolidone-vinyl acetate copolymer or dimethylaminoethyl methacrylate-butylmethacrylate-methylmethacrylate copolymer as shown in FIG. 30. No interaction in physical blend with cross-linked polyvinylpyrrolidone has been pointed out.

### 8.2 Solid State Properties of Composites

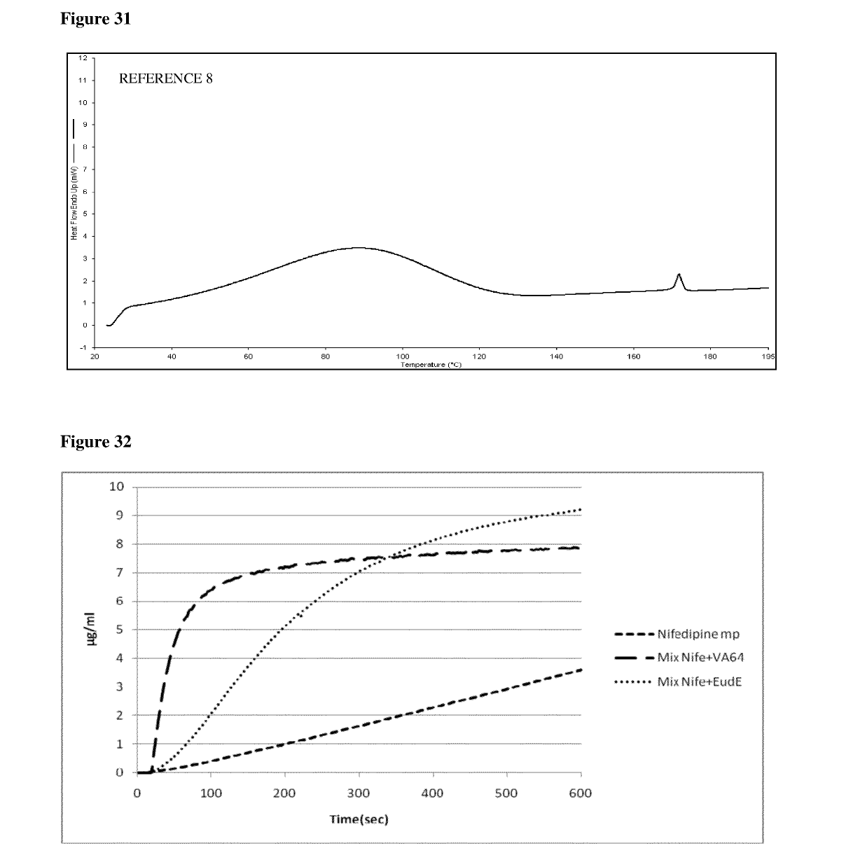

DSC scans cannot be used for evaluation of solid state properties of nifedipine into ternary composites with vinylpyrrolidone-vinyl acetate copolymer and dimethylaminoethyl methacrylate-butylmethacrylate-methylmethacrylate copolymer. DSC scan of 20% binary composite (REFERENCE 8) presented in FIG. 31 reveals presence of an aliquot of drug in crystalline form. The significant reduction of the melting enthalpy associated to nifedipine melting peak suggests that REFERENCE 8 contains both crystalline, very likely nanosized, and amorphous drug.

### 8.3 Solubilization Properties of Composites

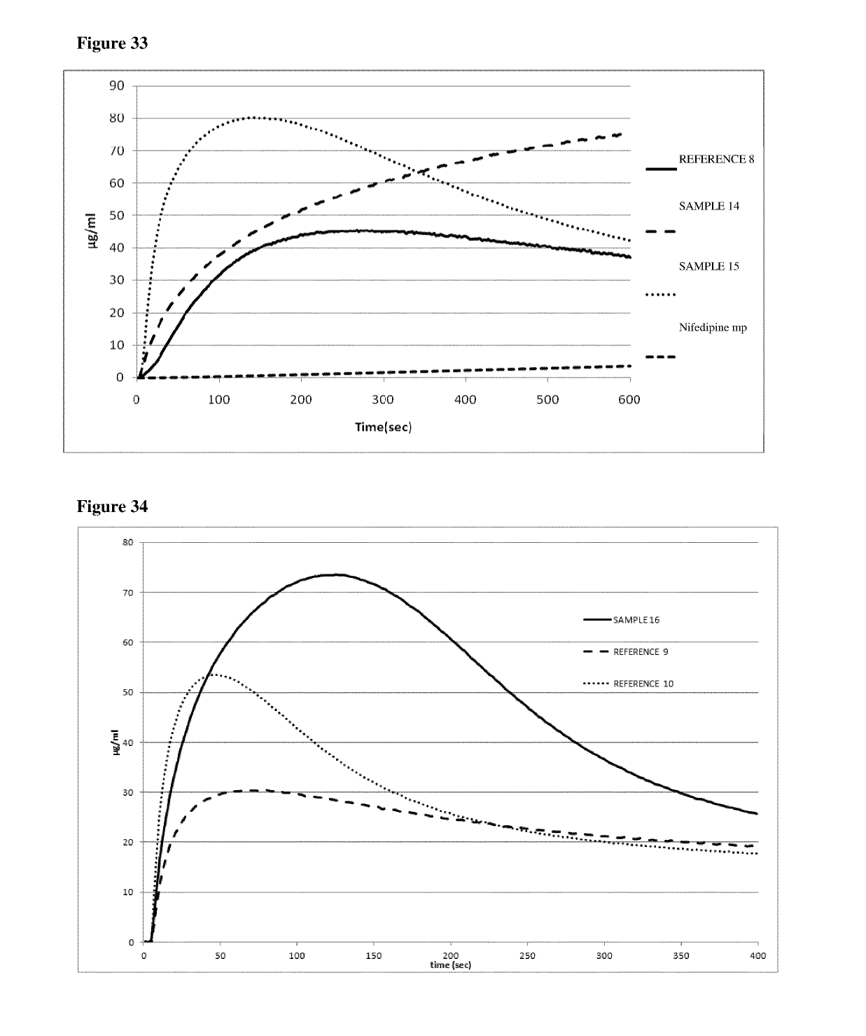

The solubilization kinetic profiles of crystalline nifedipine raw material as is and blended with each one of the investigated water and organic solvent soluble polymers (1:1 weight ratio) are presented in FIG. 32. The SK profiles of the blend is shifted upward and with different shapes respect to that of the active ingredient alone. Both vinylpyrrolidone-vinyl acetate copolymer and dimethylaminoethyl methacrylate-butylmethacrylate-methylmethacrylate copolymer promote solubilization of nifedipine (FIG. 33). Also for nifedipine the solubility performance of ternary composites (SAMPLE 14 and SAMPLE 15), is better than that of binary of equivalent drug load (REFERENCE 8).

### 9. Nimesulide Composites Characterization

### 9.1 Solid State Properties of Composites

According to evaluation of Powder X-Ray diffractograms both SAMPLE 16 and REFERENCE 9 composite seem to contain only amorphous nimesulide: no crystalline active ingredient diffraction band are found.

### 9.2 Solubilization Properties of Composites

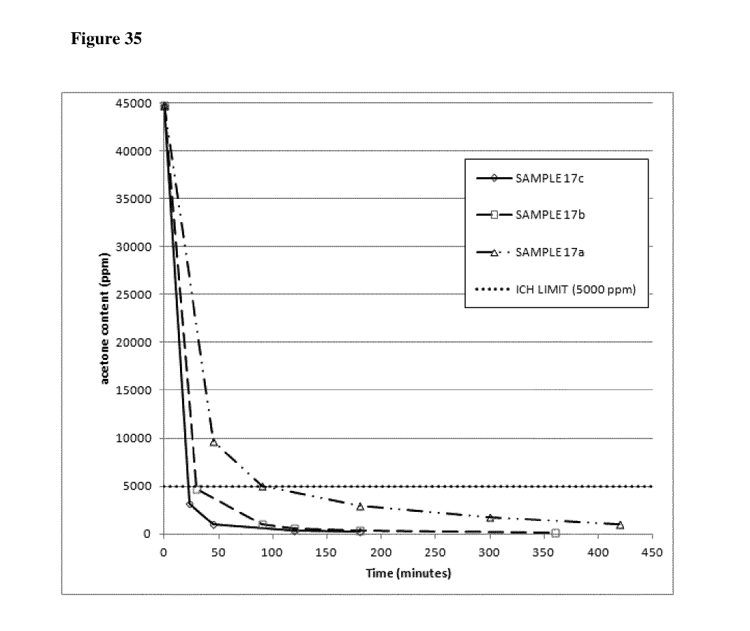

The solubilization kinetic of SAMPLE 16, REFERENCE 9 and REFERENCE 10 are presented in FIG. 34. Notwithstanding the comparable solid state of nimesulide, SK traces of SAMPLE 16 and REFERENCE 9 looks clearly different, with significantly higher solubility peak of the ternary composite (about 70 μg/ml versus about 30 μg/ml). SK trace of REFERENCE 10 is between those of SAMPLE 16 and REFERENCE 9; the solubility peak is higher than that of the binary composite, but lower than that of the ternary. This result suggests that the higher solubility performance of ternary composite is caused not exclusively by the precipitation inhibitor action (if any) of the water and organic solvent soluble polymer.

10. Composites Preparation: Effect of Solvent Removal from Swollen Composites

### 10.1 Solvent Removal: Pre-Drying Step: Granulator; Drying Step: Granulator, Vacuum Oven, Fluid Bed

The composites (SAMPLES 17 and SAMPLE 12) are prepared in a 10 L low shear mixer/granulator (Battagion IP10) at 1,800 g batch size as described in section 3.3. The end-point of the pre-drying step is fixed at LoD values of about 10% and 40% respectively for the experiments of series A (long pre-drying step of 90 minutes: SAMPLE 17) and experiments of series B (short pre-drying step of 35 minutes: SAMPLE 12). Details are given in Table 10.

At the end of the pre-drying step, three aliquots of composite of both series A and B (about 600 g as dried composite according to the LoD value of the materials) are weighed and each one is transferred into one of the three dryers selected for the drying step. The dryers and the relative drying conditions, applied in the experiment of series A and series B are as follows.

10.1.1. Low shear granulator: the heating liquid temperature is set at 55° C. until reaching a LoD % of about 3% and then was reduced to 50° C. until end of the process; vacuum applied results in pressure inside the granulator chamber of about 0.20-0.25 bar; kneading arms rotation is switched each 30 minutes to reduce formation of lumps; these drying conditions are applied for seven hours

10.1.2 Vacuum oven: the oven is pre-heated at 60° C., the sample is introduced and then heating temperature is set-up at 50° C., vacuum applied results in pressure inside the oven of about 0.25-0.30 bar; after about 120-130 minutes the drying temperature is reduced and maintained at 40° C. until end of process; drying is conducted for six hours.

10.1.3 Fluid bed (GPCG1 with insert 6″ top spray): the inlet air temperature is set at 55° C. and the air speed regulated to keep the product suspended (5.5-7.5 m/sec); humidity of the air is kept low through the connection of a dehumidifier, the air humidity is recorded in the inlet air feeding pipeline; drying time is three hours. The observed final yield at the end of the drying in fluid bed is lower than in other tested equipments because of deposition of composite on the walls of the fluid bed and on the filters.

### 10.2 Solvent Removal: Pre-Drying Step: Granulator; Drying Step: Vacuum Oven, Microwave

The composite (SAMPLE 18) comprising fenofibrate, cross-linked polyvinylpyrrolidone and vinylpyrrolidone-vinyl acetate copolymer (weight ratio 1:3:1) with drug load 20% w/w, is prepared at 150 g batch size as described in section 3.2 using low shear mixer/granulator (Battagion IP1.5/T). After completion of the homogenization, the swollen product is pre-dried for 15 minutes heating the container of the granulator at 50° C. and applying vacuum resulting in pressure inside granulator chamber of about 0.3-0.4 bar. The wet material obtained at the end of the pre-drying has a LoD of about 16.5%. Two aliquots of 45 g are taken from the pre-dried material and transferred into the two drying equipments for drying step conducted according the conditions as follows.

10.2.1. Vacuum oven at room temperature (slow drying). The pre-dried composite is distributed as a thin layer on a tray and maintained at room temperature inside an oven under vacuum (Vuototest, Mazzali) for six hours. Vacuum applied results in pressure of about 0.30 bar. Sample for LoD and GC analysis is collected after 2.5 and 5 hours and the LoD values are 5.0% and 5.1% respectively. After 2.5 hours the amount of residual acetone is about 7,600 ppm, significantly higher than the ICH guideline limit for class III solvents (5,000 ppm); after 5 hours the residual acetone is about 2,200 ppm and the drying is stopped. Sample for characterization is collected immediately after drying completion from the bulk and the remaining product is packaged into plastic bottle closed into a polyethylene bag.

10.2.2 Microwave oven (fast drying). The pre-dried composite is loaded into the container of a microwave oven with power control based on product temperature value (Microsybth model, Milestone). Drying program is applied with heating ramp to reach in 5 minutes product temperature of about 50° C., followed by isothermal step with product kept at 50° C.; total duration of this drying program is 15 minutes. The vacuum applied results in pressure inside the oven container of about 0.30 bar. Sample for characterization is collected after drying completion from the bulk and the remaining product is packaged into plastic bottle closed into a polyethylene bag. The amounts of residual solvent measured into the samples dried with the two tested methods are presented in Table 11.

TABLE 11

| | | Timepoint 1 | Timepoint 2 |
|---|---|---|---|
| Microwave | Time (min) | 15 | Not applicable |
| | Acetone | 70 | Not applicable |
| | content (ppm) | (% RSD = 5.8; | |
| | | n = 3) | |
| Vacuum oven | Time (min) | 155 | 300 |
| RT | | | |
| | Acetone | 7,591 | 2,155 |
| | content (ppm) | (% RSD = 2.9; | (% RSD = 2.4; |
| | | n = 3) | n = 3) |

It is evident that in microwave oven significantly higher amount of solvent is removed in a shorter period of time (15 minutes versus 300 minutes) than in the process under vacuum at room temperature. This confirms both the high efficiency of this drying method even for the acetone distributed within the carrier and the significant difference of solvent removal rate of the two processes.

### 10.3 Composites Characterization

The amount of crystalline drug (percentage of the drug content) found into the three aliquots of SAMPLE 17 composite dried in three different dryers is presented in Table 12.

TABLE 12

| | | Crystalline drug | Residual |
|---|---|---|---|
| | Dryer and drying | content (% of | solvent at drying |
| Sample code | duration | total drug) | end (ppm) |
| SAMPLE 17a | Low shear mixer/ | 5.68 | 1038 |
| | granulator | (stdev = 0.10) | |
| | 420 min | | |
| SAMPLE 17b | Vacuum oven | 3.31 | 166 |
| | 360 min | (stdev = 0.08) | |
| SAMPLE 17c | Fluid bed | 2.68 | 233 |
| | 180 min | (stdev = 0.06) | |

The drying by fluid bed allows to obtain a composite with lower residual crystallinity than by vacuum oven and by the low shear granulator and requires shortest time. Data presented in FIG. 35 show that the drying curve is faster and that residual solvent value below the ICH Guideline limit for acetone (<5,000 ppm) is reached earlier when the dryer is fluid bed: about 20 minutes (3,201 ppm) versus about 30 minutes of the vacuum oven (4,633 ppm) and about 180 minutes of the low shear granulator (2,951 ppm). It is also evident that different solvent removal rates are obtained with the three drying methods: in fact equivalent levels of residual solvent (about 1,000 ppm) are reached in 45, 90 and 420 minutes using fluid bed, vacuum oven and low shear granulator respectively.

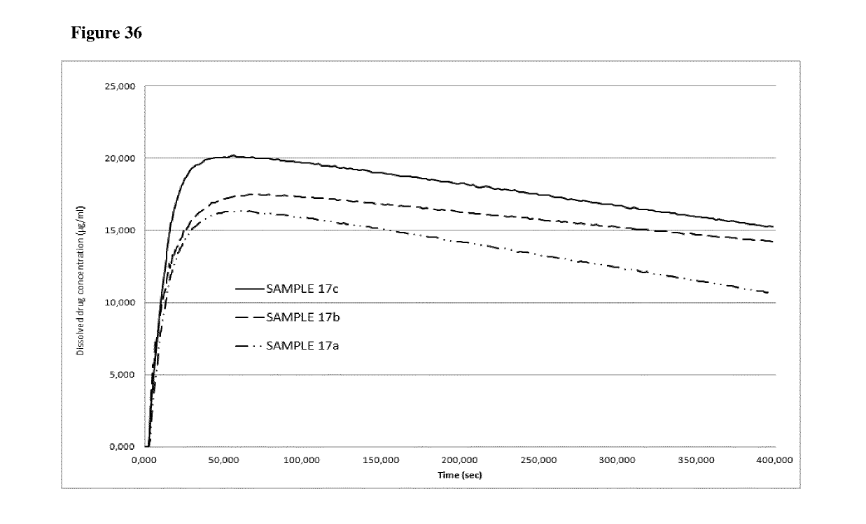

The difference of amount of crystalline drug contained in the three composites obtained with different dryers is pointed out also by the SK curves presented in FIG. 36: higher solubility level and longer precipitation time are obtained with the composite dried into the fluid bed, according to its lowest content of crystalline drug.

The amount of crystalline drug (percentage of the drug content) found into the three aliquots of SAMPLE 12 composite dried in three different dryers are presented in Table 13.

TABLE 13

| | | Crystalline | Residual |
|---|---|---|---|
| | Dryer and drying | drug content | solvent at end |
| Sample code | duration | (% of total drug) | drying (ppm) |
| SAMPLE 12a | Granulator | 6.90 | 3,988 |
| | 420 min | (stdev = 0.13) | |
| SAMPLE 12b | Vacuum oven | 0.00 | 572 |
| | 360 min | (stdev = 0.00) | |
| SAMPLE 12c | Fluid bed | 0.00 | 229 |
| | 180 min | (stdev = 0.00) | |

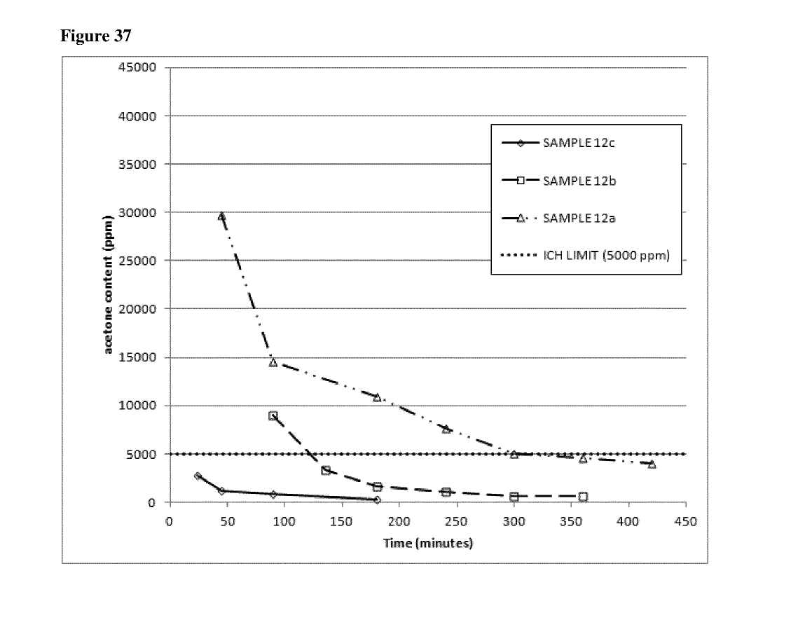

Also in this case fastest solvent removal is obtained with fluid bed dryer as shown by the drying curves presented in FIG. 37. Residual solvent level below the ICH guideline limit for acetone (5,000 ppm) is reached in about 20 minutes in fluid bed (2,802 ppm), in about 135 minutes in vacuum oven (3,289 ppm) and in about 360 minutes (4,555 ppm) in the low shear granulator. Moreover significant difference in solvent removal rate is confirmed also considering that equivalent residual solvent levels are reached in 45 minutes (1,233 ppm) and in 240 minutes (1,093 ppm) respectively in fluid bed and vacuum oven; even at the end of drying experiment (six hours), the composite dried in low shear granulator has a residual solvent level significantly higher than that of composites dried in the other two equipments (3,988 ppm).

Also in the experiment of Series B, lower crystalline drug amount are found in case of faster drying processes: both fluid bed dryer and vacuum oven allow to obtain composite containing all the drug in amorphous form. On the other hand about 7% of crystalline drug is found into the composite dried into the slowest dryer, low shear granulator. Comparing the crystalline drug content values presented in Table 13 and in Table 14 by dryer type, the effect of duration of the pre-drying step is evident. In fact, considering for example fluid bed dryer, no crystalline drug is found in the composite prepared starting from swollen material pre-dried for 35 minutes (SAMPLE 12-c), and about 2.7% crystalline drug is found in the composite prepared starting from the material pre-dried for 90 minutes (SAMPLE 17c). Pre-drying is conducted in both cases into low shear granulator used for swelling, a slow dryer; therefore longer the residence time, higher the final residual crystallinity independently from the drying process conducted. Same consideration applies to vacuum oven (SAMPLE 12b and SAMPLE 17b) and low shear granulator (SAMPLE 12a and SAMPLE 17a) dried composites.

The results of assay and quantitative solid state analysis of SAMPLES 18 are shown in Table 14.

TABLE 14

| | | Crystalline drug |
|---|---|---|
| Sample code | Drying method | (% of total drug) |
| SAMPLE 18a | Microwave oven + vacuum | 2.34 |
| | | (stdev = 0.05) |
| SAMPLE 18b | Vacuum oven RT | 23.52 |
| | | (stdev = 0.35) |

There is clear difference of solid phase distribution: ten times higher content of crystalline drug is present into the composite dried at the lowest solvent removal rate (vacuum at room temperature). The entity of this solid phase difference is significantly higher than that observed in previous experiments, this is the consequence of the enormous solvent removal rate difference between the two processes. The microwave assisted drying is here shown as very effective for acetone removal (very fast) from composites.

The experimental results (SAMPLES 12, SAMPLES 17 and SAMPLES 18) show the effect of solvent removal rate and conditions for fast pre-drying and drying.

1. A composite comprising at least one poorly water soluble drug, at least one polymeric carrier and at least one not chemically cross-linked polymer, wherein the at least one not chemically cross-linked polymer is both soluble in water and soluble in organic solvent.

2. The composite of claim 1, wherein the at least one polymeric carrier is a cross-linked polymer.

3. The composite of claim 1, wherein the at least one poorly water soluble drug is present in an amount from about 2 to about 65% by weight of the composite.

4. The composite of claim 1, wherein a weight ratio between the at least one poorly water soluble drug and the at least one polymeric carrier is from 1:0.5 to 1:50 w/w.

5. The composite of claim 1, wherein a weight ratio between the at least one poorly water soluble drug and the at least one not chemically cross-linked polymer is from 1:0.1 to 1:10.

6. The composite of claim 1, wherein the composite comprises 1 part of the at least one poorly water soluble drug, 1-18 parts of the at least one polymeric carrier, 0.5-1.5 parts of the at least one not chemically cross-linked polymer.

7. The composite according to claim 1, wherein the at least one polymeric carrier is selected from the group consisting of cross-linked polyvinylpyrrolidone, cross-linked sodium carboxymethylcellulose, cross-linked cyclodextrins, cross-linked dextran, cross-linked starch, and cross-linked methylcellulose.

8. The composite of claim 1, wherein the at least one not chemically cross-linked polymer is both soluble in the organic solvent and in water at all pH values.

9. The composite of claim 1, wherein the at least one not chemically cross-linked polymer is selected from the group consisting of hydroxypropylcellulose, hydroxyethylcellulose, hydroxypropylmethylcellulose, hydroxypropylmethycellulose acetate succinate, cellulose acetate trimellitate, acrylic and methacrylic polymers and their copolymers, methacrylic acid-methylmethacrylate copolymer, polyaminoalkyl methacrylate-methacrylic esters copolymer, dimethylaminoethyl methacrylate-butylmethacrylate-methylmethacrylatecopolymer, vinyl pyrrolidone-vinyl acetate copolymer, methylvinylether-maleic acid copolymer, and polyethyleneglycol-caprolactame-vinylpyrrolidone copolymer.

10. The composite of claim 1, wherein the at least one not cross-linked polymer is soluble both in the organic solvent and in water at a pH equal or lower than 5.

11. The composite of claim 10, wherein the at least one not chemically cross-linked polymer is dimethylaminoethyl methacrylate-butylmethacrylate-methylmethacrylate copolymer.

12. The composite of claim 1, wherein the at least one not chemically cross-linked polymer is soluble both in the organic solvent and in water at a pH equal or higher than 5.

13. The composite of claim 12, wherein the at least one not chemically cross-linked polymer is methacrylic acid-methylmethacrylate copolymer.

14. The composite of claim 1, wherein the at least one polymeric carrier is cross-linked polyvinylpyrrolidone and the at least one not chemically cross-linked polymer, which is both soluble in water and organic solvent, is selected from the group consisting of vinylpyrrolidone-vinyl acetate copolymer, and dimethylaminoethyl methacrylate-butylmethacrylate-methylmethacrylate copolymer.

15. A process for the preparation of the composite of claim 1, comprising the following steps:
1) dissolving the at least one poorly water soluble drug in a process solvent or a process solvent mixture;
2) dissolving the at least one not cross-linked polymer, which is both water and organic solvent soluble, into the drug solution of step 1);
3) swelling the at least one polymeric carrier with the solution prepared in step 2), thus obtaining a swollen composite; and
4) removing the process solvent from the swollen composite of step 3).

16. A process for the preparation of the composite of claim 1, comprising the following steps:
1-2bis) dissolving the at least one poorly water soluble drug and the at least one not cross-linked polymer, which is both water and organic solvent soluble, in a process solvent or a process solvent mixture;
3) swelling the at least one polymeric carrier with the solution prepared in step 1-2bis), thus obtaining a swollen composite; and
4) removing the process solvent from the swollen composite of step 3).

17. The process of claim 15, wherein step 3) comprises contacting of the solution of step 2) or 1-2bis) with the at least one polymeric carrier and homogeneously distributing the solution of step 2) or 1-2bis) within the mass.

18. The process of claim 15, wherein step 4) is carried out for a period of time which is equal or shorter than about 410 minutes.

19. The process of claim 15, wherein the at least one polymeric carrier is selected from the group consisting of cross-linked polyvinylpyrrolidone, cross-linked sodium carboxymethylcellulose, cross-linked cyclodextrins, cross-linked dextran, cross-linked starch, and cross-linked methylcellulose.

20. The process of claim 15, wherein the at least one not chemically cross-linked polymer, which is both soluble in water and organic solvent, is selected from the group consisting of hydroxypropylcellulose, hydroxyethylcellulose, hydroxypropylmethylcellulose, hydroxypropylmethycellulose acetate succinate, cellulose acetate trimellitate, acrylic and methacrylic polymers and their copolymers, methacrylic acid-methylmethacrylate copolymer, polyaminoalkyl methacrylate-methacrylic esters copolymer, dimethylaminoethyl methacrylate-butylmethacrylate-methylmethacrylate copolymer, vinyl pyrrolidone-vinyl acetate copolymer, methylvinylether-maleic acid copolymer, and polyethyleneglycol-caprolactame-vinylpyrrolidone copolymer.

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. A pharmaceutical composition comprising the composite of claim and pharmaceutically acceptable excipients.

35. A dosage form comprising the composite of claim 1, and pharmaceutically acceptable excipients.

36. The dosage form of claim 35 in form of tablet, capsule or orally disintegrating tablet.

37. The dosage form of claim 35 in form of sprinkle, dry syrup, extemporaneous suspension or sachets.

38. The composite of claim 1, wherein the at least one poorly water soluble drug is selected from the group consisting of fexofenadine, nifedipine, griseofulvin, indomethacin, diacerein, megestrol acetate, estradiol, progesterone, medroxyprogesterone acetate, nicergoline, clonidine, etoposide, lorazepam, temazepam, digoxin, glibenclamide ketoprofen, indobufen, ibuprofen, nimesulide, diclofenac, naproxene, acemethacine, raloxifene, paroxetine, glimepiride, anagrelide, modafanil, paroxetine, cabergoline, replaginide, glipizide, benzodiazapines, clofibrate, chlorpheniramine, digoxine, diphen-hydramine, egrotamine, estradiol, fenofibrate, griseofulvin, hydrochlothizide, hydrocortisone, isosorbide, medrogeston, oxyphenbutazone, prednisolone, prednisone, polythiazide, progesterone, spirono-lactone, tolbutamide, phenacetin, phenyloin, digitoxin, nilvadipine, diazepam, griseofulvin, and chloramphenicol.

39. The composite of claim 1, where the composite comprises the at least one poorly water soluble drug in amorphous, nano-crystalline, or both forms.
